(19) United States
(12) Patent Application Publication
(10) Pub. No.: US 2026/0028673 A1
(43) Pub. Date: Jan. 29, 2026

(54) PREFERENTIAL DEPLETION OF TCF1+ PROGENITOR T-CELLS IN SEVERE COVID-19 AS MEDIATED BY INTERLEUKIN 12 (IL-12)

(71) Applicant: Christopher Rudd, Montreal West (CA)

(72) Inventor: Christopher Rudd, Montreal West (CA)

(21) Appl. No.: 18/996,870

(22) PCT Filed: Jul. 22, 2023
(86) PCT No.: PCT/IB2023/057478

§ 371 Date: Jan. 17, 2025

Related U.S. Application Data
(60) Provisional application No. 63/391,660, filed on Jul. 22, 2022.

(51) Int. Cl. C12Q 1/6881; G01N 33/569

(52) U.S. Cl. CPC C12Q 1/6881; G01N 33/56972

(57) ABSTRACT

The coronavirus disease 2019 (COVID-19) pandemic caused by the severe acute respiratory syndrome coronavirus 2 (SARS-CoV-2) continues to be a threat to global public health. While some individuals exhibit mild symptoms, others develop severe disease leading to severe lymphopenia and death. Provided herein are methods for determining whether a subject suffering from a SARS-Cov-2 infection is more likely to suffer from severe or mild disease. Also provided are methods for predicting whether a subject suffering from a SARS-Cov-2 infection with mild disease will progress to more severe disease and methods for treating patients having an infection via SARS-Cov-2 and related family members.

### CROSS-REFERENCE TO RELATED PATENT APPLICATION

This application claims the benefit under 35 U.S.C. § 119(e) of U.S. Provisional Application No. 63/391,660, filed Jul. 22, 2022, the entire contents of which are incorporated herein by reference.

### BACKGROUND

The clinical severity of COVID-19 as mediated by the severe acute respiratory syndrome coronavirus 2 (SARS-CoV-2) ranges from asymptomatic to mild self-limiting disease to severe disease manifestations that include acute respiratory distress syndrome (ARDS), neurological symptoms and death (1-3). It is well established that the virus gains entry to host cells via SARS-CoV-2 spike(S) protein binding to the ACE2 receptors on the cell surface (4-8). However, efforts to uncover the cause(s) of poor disease outcomes have had only limited success (9-13). Clearly, there is a strong association between (SARS-CoV-2) infection and the immune system (4). Mild cases are associated with antibody response, CD4 and CD8 T-cell responses, while severe cases involve the loss of T-cells and reduced antibody responses (5-8). This is associated with increased pro-inflammatory cytokines and chemokines including IL-6 and granulocyte-macrophage colony stimulating factor (GM-CSF) (9), high neutrophil levels (9, 10), and markers of T-cell exhaustion (11). Severe infections have also been associated with decreases in regulatory T-cells (Treg) (12). The profound impact of the disease course on the immune system [11, 16, 29] and the inflammatory response has been well documented [3]. Amongst markers, the frequency of Ki67 and HLA−DR+CD38+ CD4 and CD8 T-cells increases in infected individuals (13), while severe disease is associated with the development of severe lymphopenia (14, 15). Lymphopenia has been reported to affect CD4+, CD8+ T cells, B cells and natural killer cells (16), whereas other data suggest a preferential impact on CD8+ T cells (13, 17, 18). In severe disease, lymphopenia is correlated with high levels of IL-6, IL-10 and tumour necrosis factor (TNF) (11, 15, 16, 19). These cytokines has been thought to affect T cells (19) possibly via effects on dendritic cells (20) or neutrophils (21). However, a clear notion of how activation events are linked to the loss of T-cells in severe disease remains unclear (22, 23). The respiratory tract shows a decreased contribution of cytotoxic T lymphocytes in patients with severe disease (17). This peripheral lymphopenia may partially reflect the recruitment of lymphocytes to inflamed respiratory vascular endothelium, although lung autopsy studies indicate that lymphocytic infiltration is not excessive (24-27). Other presently unknown factors likely contribute to the decline in the circulating peripheral blood.

T cell factor 1 (Tcf1, encoded by Tcf7) is a critical transcription factor and regulator of T-cell development (28). It is expressed in thymic progenitors (29), naive and effector CD4 and CD8 T-cells (30). In chronic viral infections, these self-renewing stem-like TCF1⁺ progenitors replenish the effector cell pool (31, 32). The loss of TCF1 in CD8 T cells impairs their expansion and protection against viral infections due to a loss of CD8 memory precursors (33). TCF1+ expressing memory-like CD8(+) T cells sustain the immune response against LCMV (34) and cytomegalovirus infections (35). Similarly, TCF1+ cells are needed for successful cancer immunology with immune checkpoint inhibitors (34, 36-39). The genetic knockout of Tcf7 in CD8⁺ T cells reduces both the number and polyfunctionality of memory-precursor-like T cells (40). The memory-precursor-like PD-1 negative CD8⁺ TCF1^{hi} T cells are responsible for long-lasting anti-tumor responses (40). TCF1 can also define a subset of progenitor exhausted T-cells (41).

Other factors also affect the proliferation, function and viability of T-cells needed to mount and to sustain an immune response against foreign antigen, viral infections, and cancer. Ki67 is a well-established marker for cell cycling and proliferation since it down-regulated in resting G0 cells (42). As mentioned, its expression is widely used as a measure of the proliferation index that determines treatment decisions (43). The pro-survival mediator BcL2 is a founding member of a family of mediators that control cell survival (44). Other markers for progenitor cells include LEF1 (for lymphoid enhancer-binding factor), Notch1 and ZEB1 (TCF8). LEF-1 (for lymphoid enhancer-binding factor) interacts with beta-catenin (45), while the combined effects of TCF1 and LEF1 provide constant supervision of the differentiation states of CD8+ T-cells (46). Similarly, Notch 1 signaling is required for commitment of thymic T-cell progenitors, promoting proliferation and survival (47). ZEB represses T-lymphocyte-specific interleukin 2 expression by binding to a negative regulatory domain next to the IL-2 transcription start site (48).

### SUMMARY OF THE DISCLOSURE

Applicant identified several markers that allows one to distinguish disease severity in patients with COVID-19. This includes transcription factors (i) TCF-1 (Tcf7), (ii) for lymphoid enhancer-binding factor (LEF-1), (iii) transcription factor Eomes, (iv) the progenitor marker Notch as well as the (iv) cell cycle protein Ki-67, (v) pro-survival factor Bcl-2 and (vi) the pro-apoptotic caspase 3 in peripheral T-cells of the immune system. The reduced expression of TCF1 (Tcf7) and Ki67 is correlated with disease severity. The lower level of expression is correlated with an increased severity of disease as measured using the World Health Organization (WHO)'s ordinal scale (WOS) for disease severity 3-8. The reduced expression of pro-survival factor Bcl-2 also correlated with disease severity. The grouping of patients with mild (WOS 3-4) and severe (WOS 3-8) disease also showed a statistical difference with the reduced expression of TCF1, Ki67 and Bcl2 in T-cells from severe patients. The loss of Ki67 expression is indicative of a loss in the proliferation of the TCF1+ progenitor T-cell population that is needed to replenish the immune system for a response to infection. Further, the expression of other progenitor factors, LEF-1 and Notch 1 is also statistically reduced in CD8+ T-cells. Consistent with the reduced expression of Ki67 and Bcl2, Applicant also observed an increase in the expression of pro-apoptotic caspase 3 in peripheral T-cells in severe disease amongst CD8+ T-cells indicative to cell death. Also observed was a decrease in the expression of Eomes in T-cells from patients with severe disease. The altered expression of these factors was seen by flow cytometry and by qPCR analysis.

Utilizing these observations, the combination of these markers is the most effective measure of the transition to disease severity. It can predict whether patients with mild-moderate disease will progress to life-threatening severe COVID-19. It can also be applied to the diagnosis of disease severity to other conditions, such as in response to infection by other viruses such as variants of SARS-CoV2, other human coronaviruses (e.g., 229E, NL63, OC43, HKU1, MERS-CoV, or SARS-CoV), Dengue, Hep A and B. The combination of the disclosed markers can also be used to monitor the status and progression of disease in a subject infected with SARS-CoV2, variants of SARS-CoV2, other human coronaviruses (e.g., 229E, NL63, OC43, HKU1, MERS-CoV, or SARS-CoV), Dengue, Hep A or Hep B and in one aspect, direct treatment decisions. For example, use of the markers can identify those patients more likely to experience moderate or severe COVID, and an appropriate therapy can be prescribed by the treating physician.

Without being bound by theory, Applicant identified that reduced expression of TCF-1 (Tcf7) can be explained by the proinflammatory cytokine interleukin 12 (IL-12). Sera from several patients showed down-regulated TCF1 expression, which in turn could be prevented by the use of blocking antibodies to IL-12. Thus, the use of anti-IL-12 blocking antibodies such as STELARA® (ustekinumab, a monoclonal antibody developed by Janssen Pharmaceuticals), presently used to treat psoriasis and Crohn's disease (CD) (49) can also prevent the loss of T-cells in severe disease. Further, in this context, the anti-inflammatory glucocorticoid, dexamethasone, which has proven successful in treating COVID-19 patients (50), also inhibits IL-12 production (51). In addition, the disclosed data shows that the combination can diagnose and possibly predict disease followed by the treatment of patients with anti-IL 12.

### BRIEF DESCRIPTION OF THE FIGURES

FIGS. 1A-1L: The loss of T-cells in patients with mild and severe COVID-19. Peripheral T-cells were extracted from healthy donors and patients with mild or severe disease followed by an analysis of surface receptors by flow cytometry. (FIG. 1A) Percent of T-cells with the peripheral blood mononuclear population. Upper panel: histogram showing the loss of T-cells in mild and severe patients; lower panel: Spearman analysis showing the loss of T-cell relative severity score. (FIG. 1B) Percent of CD4+ T-cells with the peripheral blood mononuclear population. Upper and lower panels as in (FIG. 1A). (FIG. 1C) Percent of CD8+ T-cells with the peripheral blood mononuclear population. Upper and lower panels as in (FIG. 1A). (FIG. 1D) Percent of TCR-beta T-cells with the peripheral blood mononuclear population. (FIG. 1E) Percent of TCR-gamma/delta T-cells with the peripheral blood mononuclear population. (FIG. 1F) Percent of CD69 T-cells with the CD4 and CD8+ peripheral T-cells. Upper panel: CD4+ T-cells; lower panel: CD8+ T-cells. (FIG. 1G) Percent of PD-1+ T-cells with the CD4 and CD8+ peripheral T-cells. Upper panel: CD4+ T-cells; lower panel: CD8+ T-cells. (FIG. 1H) Percent of Notch 1 T-cells with the CD4 and CD8+ peripheral T-cells. Upper panel: CD4+ T-cells; lower panel: CD8+ T-cells. (FIG. 1I) Percent of GZMB+ CD8+ peripheral T-cells. Upper panel: percentage; lower panel: Spearman analysis showing a correlation between GZMB expression and disease severity. (FIG. 1J) Percent of IFNg+ CD4+ peripheral T-cells. Upper panel: CD4+ T-cells; Lower panel: Spearman analysis showing a correlation between IFN-g expression and disease severity. (FIG. 1K) Percent of IFNg+ CD8+ peripheral T-cells. Upper panel: CD8+ T-cells; Lower panel: Spearman analysis showing a correlation between IFN-g expression and disease severity.

FIGS. 2A-2D: A comparison in the loss of TCF+ CD8 and CD4+ peripheral T-cells in severe patient versus mild patients as correlated with increasing disease severity. Peripheral T-cells were extracted from healthy donors and patients with mild or severe disease followed by an analysis of surface receptors by flow cytometry. (FIG. 2A) The preferential loss of CD8+TCF1+ cells in blood of human patients with COVID disease severity. Upper panel: histogram showing the loss of CD8+TCF1+ peripheral T-cells cells in severe patient versus mild patients. Patients were grouped into mild (WOS 2-4) or severe (WOS 5-8). Lower panel: histogram showing the loss of CD8+ TCF1+ peripheral T-cells with increasing severity of COVID-19 disease (Spearman analysis). (FIG. 2B) CD8+TCF1− CD8+ T-cells in blood of human patients show no significant loss (same as in FIG. 2). Upper panel: histogram showing the absence of loss of CD8+TCF1+ cells in severe patients. Lower panel: histogram showing no correlation in the expression of CD8+ TCF1+ peripheral T-cells with the increasing severity of COVID-19 disease (Spearman analysis). (FIG. 2C) The preferential loss of CD4+TCF1+ cells in blood of human patients with COVID disease severity. Upper panel: histogram showing the loss of CD4+TCF1+ peripheral T-cells cells in severe patients versus mild patients. Lower panel: histogram showing the loss of CD4+ TCF1+ with the increasing severity of COVID-19 disease (Spearman analysis). (FIG. 2D) CD4+TCF1− T-cells in blood of human patients show no significant loss (same as in FIG. 2). Upper panel: histogram showing the absence of loss of CD4+TCF1+ cells in severe patients. Lower panel: histogram showing no correlation in the expression of CD4+ TCF1+ peripheral T-cells with increasing severity of COVID-19 disease (Spearman analysis).

FIGS. 3A-3K: Selective loss of TCF+ T-cells in severe COVID-19. Peripheral T-cells were extracted from healthy donors and patients with mild or severe disease followed by an analysis of surface receptors by flow cytometry. (FIG. 3A) The preferential loss of CD8+TCF1+ cells in blood of human patients. Upper panel: histogram showing the loss of CD8+TCF1+ cells in severe patients; lower panel: Spearman analysis showing a correlation between CD8+TCF1+ cell number and disease severity. (HD (n=5), Mild (n=5), Severe (n=7)). (FIG. 3B) The presence of CD8+TCF1− cells in blood of human patients. Upper panel: histogram showing the continued presence of CD8+TCF1− cells in severe patient; lower panel: Spearman analysis showing no correlation between CD8+TCF1+ cell number and disease severity (HD (n=5), Mild (n=5), Severe (n=7)). (FIG. 3C) Histogram showing the loss of TCF1+ peripheral T-cells in the CD8 subset in severe patients relative to mild patients. Grouped as percentages in mild (WOS 2-4) or severe (WOS 5-8) (HD (n=10), Mild (n=32), Severe (n=31)). (FIG. 3D) Histogram showing the loss of TCF1+ peripheral T-cells in the CD4 subset in severe patients relative to mild patients. Grouped as percentages in mild (WOS 2-4) or severe (WOS 5-8) (HD (n=10), Mild (n=32), Severe (n=31)). (FIG. 3E) Histogram showing the fold change of the MFI in the expression of TCF1+ in CD8+ T-cells from patients with severe disease relative to mild disease. Values are expressed as a fold change of the MFI of peripheral T-cells in the CD8 subset. (FIG. 3F) Spearman analysis of the loss of TCF1+ expression as expressed as a fold change of the MFI relative to the WOS score (r=−0.447; p=*). (FIG. 3G) viSNE patterns of anti-CD8, CD4, CD44 and TCF1 staining of T-cells from the peripheral blood of HD, mild and severe patients. Upper panel: density pattern with 10 clusters of cells; lower panel: histogram showing the total expression of TCF1 in islands 1, 2 3, 4, 5, 6, 7, 8, 9, and 10 from HD, mild and severe. (FIG. 3H) viSNE patterns of TCF1 staining of CD8 T-cells from the peripheral blood of HD, mild and severe patients. Upper panel: density pattern with 3 clusters of cells based on densiometric analysis showing groupings of cells (left panel) and the expression of TCF1 (right panel). Lower panel: histogram showing the total expression of TCF1 in islands 1, 2 and 3, from HD, mild and severe. (FIG. 3I) SPADE patterns showing the loss of TCF1 expression in multiple tree groupings found in severe vs mild patients for CD8+ T-cells. SPADE analysis identified over 100 subsets or nodes with relative degrees of similarity seen in each tree branch (fluorescence intensity of the different markers for each node are represented by color while the size of the node represents the number of cells). The figure shows the concatenated samples from 9 patients in each treatment group involving multiple markers (12 different antibodies). Equal numbers of cells per treatment group were analyzed; branches ii-iv in mild and v-vii in severe. The pattern in severe disease showed an increase in the number of nodes with reduced TCF1 expression (dark blue-green color). (FIG. 3J) viSNE patterns of CD4+ T-cells from the peripheral blood of HD, mild and severe patients. Upper panel: histogram showing the total expression of TCF1 in islands 1-3 from HD, mild and clusters 4, 5 in pattern for severe patients. Upper panel: viSNE patterns. Lower panel: histogram. (FIG. 3K) SPADE patterns showing the loss of TCF1 expression in multiple tree groupings found in severe vs mild patients for CD4+ T-cells. Equal numbers of cells per treatment group were analyzed; branches ii-iv in mild and v-vii in severe. The TCF1+ CD4 cells were found in clusters iv-v in HD and mild patients, while severe disease showed an increase in the number of nodes v-viii with reduced TCF1 expression (light link and dark blue-green color).

FIGS. 4A-4B: Loss of TCF+ T-cells at differential stages of differentiation in severe COVID-19. Peripheral T-cells were extracted from healthy donors and patients with mild or severe disease followed by an analysis of surface receptors by flow cytometry. (FIG. 4A) Severe disease is accompanied by a loss of TCF1+ CD8+ naïve (CCR7+CD45RA+), central memory (TCM, CCR7+CD45RA−), effector memory (TEM, CCR7−CD45RA−), and effector memory cells re-expressing CD45RA subset (TEMRA, CCR7−CD45RA+). (FIG. 4B) Histogram showing the relative loss of TCF1+ in KLRG1+CD127− and KLRG1+CD127+, KLRG1-CD127+ and KLRG1-CD127− CD8+ T-cells in T-cells from severe patients.

FIG. 5A-5B: The loss of other progenitor factors such as LEF1 and Notch 1 in T-cells with severe disease. Peripheral T-cells were extracted from healthy donors and patients with mild or severe disease followed by an analysis of surface receptors by flow cytometry. CD3+ T-cells were gated into CD4+ and CD8+ T-cells from analysis. The expression of LEF1, Notch1, ZEB1 and TCF1 was analyzed in CD8+ T-cells (FIG. 5A) and CD4+ T-cells (FIG. 5B).

FIG. 6A-6F: Longitudinal analysis of TCF+ T-cells with mild to severe COVID-19. Samples were first taken from patient CC122 infected with SARs CoV2 with severity ranking of 4 followed by 9 days and a second sample extraction when the patient had a severity score of 6. Peripheral T-cells were extracted and assessed for TCF1 expression on T-cells, CD4 and CD8 T-cells. (FIG. 6A) Relative frequency of TCF1+ T-cells (upper panel), TCF1+CD4+ T-cells (middle panel) and TCF1+ CD8+ T-cells (lower panel) in peripheral blood. (FIG. 6B) Mean fluorescent intensity (MFI) of TCF1 on T-cells (upper panel); CD4+ T-cells (middle panel) and CD8+ T-cells (lower panel). (FIG. 6C) ViSNE analysis showing the loss of TCF1 expression in CD4 (left panel) and CD8+ (right panel) T-cells from patients with severity 4 and 6. Lower histograms showing total TCF expression (MFI x numbers of cells) in each of the clusters. CD4+ T-cells (left lower histogram) and CD8+ T-cells (lower right histogram). (FIG. 6D) SPADE patterns showing the presence of subsets (nodes) of CD4+ TCF1+ cells longitudinally from a WOS severity ranking of 4 (left grouping) to 6 (right grouping). Based on antibody staining, SPADE analysis identified over 100 subsets or nodes with relative degrees of similarity seen in each tree branch. The fluorescence intensity of the different markers for each node is represented by color while the size of the node represents the number of cells. Within each pattern, Applicant focused on 5 groupings of tree clusters and their nodes (i-v). Left panel: severity 4 with TCF1 expression found in tree cluster iii. Right panel: severity 6 with reduced TCF1 expression found in tree cluster iv. (FIG. 6E) SPADE patterns showing the presence of subsets (nodes) of CD8+TCF1+ cells longitudinally from a WOS severity ranking of 4 (left grouping) to 6 (right grouping). Patterns show a shift in the numbers of nodes with high to low TCF1 expression). Left panel: severity 4 with TCF1 expression found in tree clusters iii and v. Right panel: severity 6 with reduced TCF1 expression and cell numbers on nodes found in tree cluster iv. (FIG. 6F) Spearman analysis of the decrease in TCF1+ CD8+ T-cells (fold change) relative to disease severity (f=−0.8982; p=0.0095) .

FIG. 7A-7N: Selective depletion of Ki67+ T-cells in severe COVID-19. Peripheral T-cells were extracted from healthy donors and patients with mild or severe disease followed by an analysis of surface receptors by flow cytometry. (FIG. 7A) The preferential loss of the percent of Ki67+ peripheral T-cells in the CD8 subset as grouped into mild (WOS 2-4) or severe (WOS 5-8) (HD (n=12), Mild (n=36), Severe (n=34)). (FIG. 7B) The preferential loss of the percent of Ki67+ peripheral T-cells in the CD4 subset as grouped into mild (WOS 2-4) or severe (WOS 5-8) (HD (n=12), Mild (n=36), Severe (n=34). (FIG. 7C) viSNE patterns of anti-Ki67 staining of TCR-beta+ T-cells from the peripheral blood of HD, mild and severe patients. Histogram showing the total expression of Ki67 in different clusters of cells from HDs and patients with mild or severe disease. Anti-TCF1 and interferon-gamma staining is also shown. Lower panels show histograms for Ki67 (left panel) and IFN-gamma (right panel) expression. (FIG. 7D) viSNE analysis of Ki67 expression in CD8+ T-cells from patients with mild vs. severe disease. This is a representative pattern from a single patient. Overall analysis was done on >15 patients in each group. Lower histogram showing the mean of Ki67 expression in clusters 1-3. (FIG. 7E) SPADE patterns showing the presence of subsets (nodes) of CD8+ Ki67+ T-cells in HDs. SPADE analysis identified over 100 subsets or nodes. Within each pattern, Applicant focused on 8 groupings of tree clusters and their nodes (i-viii). (FIG. 7F) SPADE patterns showing the presence of subsets (nodes) of CD8+ Ki67+ T-cells from patients with mild disease (concatenated 8 patients). (FIG. 7G) SPADE patterns showing the presence of subsets (nodes) of CD8+ Ki67+ T-cells with severe disease (concatenated 8 patients). (FIG. 7H) SPADE patterns showing the presence of subsets (nodes) of CD8+TCF1+ T-cells with severe disease (concatenated 8 patients). (FIG. 7I) viSNE analysis of Ki67 expression in CD4+ T-cells from patients with mild vs. severe disease. This is a representative pattern from a single patient. Overall analysis was done on >15 patients in each group. Lower histogram showing the mean of Ki67 expression in clusters 1-3. (FIG. 7J) SPADE patterns showing the presence of subsets (nodes) of CD4+ Ki67+ T-cells in peripheral cells from HDs (concatenated 8 patients). (FIG. 7K) SPADE patterns showing the presence of subsets (nodes) of CD4+ Ki67+ T-cells in peripheral cells from patients with mild disease (concatenated 8 patients). (FIG. 7L) SPADE patterns showing the presence of subsets (nodes) of CD4+ Ki67+ T-cells in peripheral cells from patients with severe disease (concatenated 8 patients). (FIG. 7M) SPADE patterns showing the presence of subsets (nodes) of CD4+ Ki67+ T-cells in peripheral cells from patients with severe disease (concatenated 6 patients). (FIG. 7N) SPADE patterns showing the presence of subsets (nodes) of CD4+ TCF1+ T-cells in peripheral cells from patients with severe disease. For comparison with panel M, this pattern shows a heterogeneous collection of nodes with different levels of TCF1+ expression (light blue to green and yellow) as well as several nodes lacking TCF1 (dark blue). The loss of Ki67 expression overlaps with nodes expressing different levels of TCF1 (see arrows comparing panel M to N).

FIGS. 8A-8J. Cells with reduced TCF1+ expression show reduced pro-survival BcL-2 combine with the appearance of a new subset with high caspase 3 indicative of cell death. (FIG. 8A) The loss of the percent of Bcl-2+ peripheral CD8+ T-cells in patients with severe disease. Samples grouped into mild (WOS 2-4) or severe (WOS 5-8) (HD (n=4), Mild (n=18), Severe (n=15)). (FIG. 8B) viSNE analysis of a representative sample of cells taken from HDs and patients with mild or severe disease. Pattern shows an increase in Bcl2 expression in mild patient followed by a loss in severe patients. Right panel histogram shows a reduction in Bcl2 expression in different clusters. (FIG. 8C) Conventional flow cytometry showing the expression of SLAMF6, Caspase 3, PD1 and TIM3 on CD8+ T-cells. In keeping with TCF1 expression, SLAMF6 expression was reduced in T-cells accompanied by the appearance of peaks of high caspase 3 staining in samples from severe patients. Lower heat map shows the increase in overall caspase 3 staining, the loss of SLAMF6 expression and an increased in expression of PD1 and TIM3. (FIG. 8D). viSNE patterns of anti-SLAMF8 and anti-caspase 3 in CD8+ T-cells. Left panel: SLAMF6 expression in islands 1, 2, and 3. Right panels: Caspase 3 expression in islands 1, 2, and 3. Lower histogram showing the MFI of expression of caspase 3 in severe patients. (FIG. 8E) SPADE patterns showing the presence of subsets (nodes) of CD8+ caspase 3+ T-cells from HDs (concatenated 8 patients). (FIG. 8F) SPADE patterns showing the presence of subsets (nodes) of CD8+ caspase 3+ T-cells from patients with mild disease (concatenated 8 patients). (FIG. 8G) SPADE patterns showing the presence of subsets (nodes) of CD8+ caspase 3+ T-cells from patients with severe disease (concatenated 8 patients). Circled cells show the appearance of a population of Caspase 3 high expressing cells. (FIG. 8H) SPADE patterns showing the presence of subsets (nodes) of CD8+ SLAMF6+ T-cells from patients with severe disease (concatenated 8 patients). Circled cells show the appearance of a population which has lost TCF1 expression. (FIG. 8I) Ex vivo cultures of T-cells from HDs and patients with mild or severe disease. Peripheral T-cells were cultured for 6 days followed by an assessment of cell viability. (FIG. 8J) Ex vivo cultures of T-cells from HDs and patients with mild or severe disease show a sustained loss of the transcription of TCF1 (upper left panel); Ki67 (upper right panel); Bcl2 (lower left panel) and LEF1 (lower right panel). Peripheral T-cells were cultured for 6 days followed by an assessment of expression by qPCR.

FIGS. 9A-9D: Sera from severe patients suppresses the expression of TCF1 in an IL-12 dependent manner. Sera from healthy donors or severe patients was added to normal T-cells from peripheral blood and incubated for several days as outlined in the Methods. (FIG. 9A) Percent TCF1+ T-cells in the CD8+ subsets showed reduced TCF1 expression reversed by co-incubation with anti-IL12. (FIG. 9B) Percent TCF1+ T-cells in the CD4+ subsets showed reduced TCF1 expression reversed by co-incubation with anti-IL12. (FIG. 9C) ViSNE patterns showing the loss of TCF1 expression due to incubation with severe sera (top and second from the bottom) (see clusters 1, 2, and 3). Anti-IL12 co-incubation restored TCF1 expression (see left histograms). (FIG. 9D) Severe sera reduced TCF 1 expression in all subsets of T-cells (naïve, TCM, TEM and TEMRA) which was restored by co-incubation with IL-12.

FIG. 10 is a summary of the embodiments of this disclosure. Applicant discovered that the transition from mild to severe COVID-19 is characterized by the preferential reduction of TCF1+ and Ki67 expression in CD4+ and CD8+ T-cells in patients with severe disease. This in turn correlates with the preferential loss of TCF1+ T-cells relative to TCF1-T-cells in the peripheral blood of patients with severe disease. A comparison of healthy donors with patients with mild disease showed an increase in the expression of Ki67 on TCF1+ and TCF1− T-cells indicative of proliferation and an immune response against SARS CoV2. However, a marked loss of Ki67 was seen in TCF1+ and TCF1−CD4+ and CD8+ T-cells from patients with severe disease. A loss in other markers was also observed such as the progenitor transcription factor EOMES in T-cells, as well as LEF1 and the progenitor marker Notch 1 in CD8+ T-cells. The loss of proliferation of the progenitor stem-like population is needed to replenish the immune system. This transition from mild to severe disease was further accompanied by a reduction in the expression of pro-survival BcL2 resulting in the final stage of an increase in caspase 3 expression (cell death) especially in the TCF1-cells. The loss of these factors was seen by flow cytometry and the quantitative PCR. Without being bound by theory, this is why the immune system collapses with a large loss of T-cells and mortality.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the disclosure to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the disclosure. All publications, patent applications, patents and other references mentioned herein are incorporated by reference in their entirety.

Throughout this disclosure, technical and patent references are identified by an Arabic numeral or citation. These are provided to describe the state of the art. The full bibliographic citation for technical and patent references can be found immediately preceding the claims.

Unless the context indicates otherwise, it is specifically intended that the various features of the disclosure described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications are referenced by an identifying citation or by an Arabic numeral. The full citations for the publications identified by an Arabic numeral are found immediately preceding the claims. The disclosures of these publications, patents and published patent specifications are hereby incorporated by reference into the present disclosure in their entirety to more fully describe the state of the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the description of the disclosure and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

As used herein, the term “comprising” is intended to mean that the compositions or methods include the recited steps or elements, but do not exclude others. “Consisting essentially of” shall mean rendering the claims open only for the inclusion of steps or elements, which do not materially affect the basic and novel characteristics of the claimed compositions and methods. “Consisting of” shall mean excluding any element or step not specified in the claim. Embodiments defined by each of these transition terms are within the scope of this disclosure.

The terms “acceptable,” or “effective,” or “sufficient,” when used to describe the selection of any components, ranges, dose forms, etc. disclosed herein, intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “animal” refers to living multi-cellular vertebrate organisms, a category that includes, for example, mammals and birds. The term “mammal” includes both human and non-human mammals.

The terms “subject,” “host,” “individual,” and “patient” are as used interchangeably herein to refer to animals, typically mammalian animals. Any suitable mammal can be treated by a method, cell or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a cancer or neoplastic disorder.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human,

“Prokaryotic cells” that usually lack a nucleus or any other membrane-bound organelles and are divided into two domains, bacteria and archaea. In addition to chromosomal DNA, these cells can also contain genetic information in a circular loop called an episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “composition” typically intends a combination of the active agent, e.g., the nanoparticle of this disclosure and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions used in accordance with the disclosure, including cells, treatments, therapies, agents, drugs and pharmaceutical formulations can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to the number of treatments and unit dose, depends on the result and/or protection desired. Precise amounts of the composition also depend on the judgment of the practitioner and are peculiar to each individual. Factors affecting the dose include the physical and clinical state of the subject, the route of administration, the intended goal of treatment (alleviation of symptoms versus cure), and the potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “isolated cell” generally refers to a cell that is substantially separated from other cells of a tissue. The term includes prokaryotic and eukaryotic cells.

An “effective amount” or “efficacious amount” refers to the amount of an agent or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated. In some embodiments the effective amount will depend on the size and nature of the application in question. It will also depend on the nature and sensitivity of the target subject and the methods in use. The skilled artisan will be able to determine the effective amount based on these and other considerations. The effective amount may comprise, or alternatively consist essentially of, or yet further consist of one or more administrations of a composition depending on the embodiment.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

As used herein, the term “an acute respiratory syndrome coronavirus 2” (SARS-Cov-2 or SARS disease) refers to is a strain of coronavirus that causes COVID-19 (coronavirus disease 2019), the respiratory illness responsible for the ongoing COVID-19 pandemic. It is a positive-sense single-stranded RNA (+ssRNA) virus, with a single linear RNA segment. SARS-CoV-2 is a member of the subgenus Sarbecovirus (beta-CoV lineage B). Like other coronaviruses, SARS-CoV-2 has four structural proteins, known as the S (spike), E (envelope), M (membrane), and N (nucleocapsid) proteins; the N protein holds the RNA genome, and the S, E, and M proteins together create the viral envelope.

As used herein, the term “severe SARS disease” refers to a case of SARS disease wherein the case has a World Health Organization (WHO) severity score of 5 or higher.

As used herein, the term “mild SARS disease” refers to a case of SARS disease wherein the case has a World Health Organization (WHO) severity score of 4 or lower.

As used herein, the term “transcription factor TCF1+” (T-cell factor-1) refers to the T cell factor 1 (Tcf1, encoded by Tcf7) which is a key transcription factor of the human immune system. TCF1 is an essential transcription factor for early T cell development. TCF1 is required for the self-renewal of stem-like CD8⁺ T cells generated in response to viral or tumour antigens, and for preserving heightened responses to checkpoint blockade immunotherapy.

As used herein, the term “lymphoid enhancer-binding factor” (LEF-1) refers to a 48-kD nuclear protein that is expressed in pre-B and T cells. LEF-1 binds to a functionally important site in the T-cell receptor-alpha enhancer and confers maximal enhancer activity.

As used herein, the term “transcription factor Eomesodermin/Tbr2” (Eomes) refers to a transcription factor which controls gene expression involved in the regulation of developmental processes. Eomesodermin/Tbr2 itself controls regulation of radial glia, as well as other related cells. More importantly for this disclosure, Eomesodermin/Tbr2 has also been found to have a role in immune response, and there exists some evidence for its connections in other systems

As used herein, the term “progenitor marker Notch” (Notch) refers to a family of type-1 transmembrane proteins that form a core component of the Notch signaling pathway. The Notch intracellular domain acts as a transcriptional activator when in complex with CSL family transcription factors.

As used herein, the term “cell cycle protein Ki-67” (Ki-67) refers to a nuclear protein that is associated with cellular proliferation. Ki-67 protein is present during all active phases of the cell cycle (G₁, S, G₂, and mitosis), but is absent in resting (quiescent) cells (G₀). Cellular content of Ki-67 protein markedly increases during cell progression through S phase of the cell cycle.

As used herein, the term “pro-survival factor Bcl-2” (Bcl-2) refers to a regulator protein that regulates cell death (apoptosis), by either inhibiting (anti-apoptotic) or inducing (pro-apoptotic) apoptosis. BCL-2 is localized to the outer membrane of mitochondria, where it plays an important role in promoting cellular survival and inhibiting the actions of pro-apoptotic proteins.

As used herein, the term “pro-apoptotic caspase-3” (caspase-3) refers to a caspase protein that interacts with caspase-8 and caspase-9. Sequential activation of caspases plays a central role in the execution-phase of cell apoptosis.

As used herein, the term “T cells” refers to a type of lymphocyte. T cells are one of the important white blood cells of the immune system and play a central role in the adaptive immune response. Groups of specific, differentiated T cell subtypes have a variety of important functions in controlling and shaping the immune response. One of these functions is immune-mediated cell death, and it is carried out by two major subtypes: CD8+ “killer” and CD4+ “helper” T cells. (These are named for the presence of the cell surface proteins CD8 or CD4.) CD8+ T cells, also known as “killer T cells”, are cytotoxic—this means that they are able to directly kill virus-infected cells, as well as cancer cells.

As used herein, the term “biological sample” refers to any sample retrieved from a subject that contains T-cells that can be measured by the methods described herein. Exemplary samples include, but are not limited to, peripheral blood mononuclear cells (PBMCs) whose analysis are described by the methods herein.

As used herein, the term “lymphopenic” refers to the condition of having an abnormally low level of lymphocytes in the blood. Lymphocytes are white blood cells with important functions in the immune system.

As used herein, the term “peripheral blood lymphocytes” (PBMCs) refers to, but is not limited to, lymphocytes (T cells, B cells, and NK cells), monocytes, and dendritic cells.

As used herein, the term Interleukin 12 (IL-12) refers to an interleukin that is naturally produced by dendritic cells, macrophages, neutrophils, and human B-lymphoblastoid cells in response to antigenic stimulation. IL-12 plays an important role in the activities of natural killer cells and T lymphocytes. IL-12 mediates enhancement of the cytotoxic activity of NK Cells and CD8+ cytotoxic T lymphocytes.

As used herein, the term “glucocorticoid” refers to a class of corticosteroids, which are a class of steroid hormones. Glucocorticoids are corticosteroids that bind to the glucocorticoid receptor. Glucocorticoids are part of the feedback mechanism in the immune system, which reduces certain aspects of immune function, such as inflammation. They are therefore used in medicine to treat diseases caused by an overactive immune system, such as allergies, asthma, autoimmune diseases, and sepsis.

As used herein, the term “DNA microarrays” refers to a collection of microscopic DNA spots attached to a solid surface. DNA microarrays may be used DNA microarrays to measure the expression levels of large numbers of genes simultaneously or to genotype multiple regions of a genome.

As used herein, the term “Real-time polymerase chain reaction” (Real-time PCR) refers to the amplification of a targeted DNA molecule during the PCR, not at its end, as in conventional PCR. Real-time PCR may be used quantitatively (quantitative real-time PCR) and semi-quantitatively (semi-quantitative real-time PCR).

As used herein, the term “Chromatin immunoprecipitation” (ChIP) refers to a type of immunoprecipitation experimental technique used to investigate the interaction between proteins and DNA in the cell. ChIP determines whether specific proteins are associated with specific genomic regions, such as transcription factors on promoters or other DNA binding sites.

As used herein, the term “flow cytometry” refers to a technique used to detect and measure physical and chemical characteristics of a population of cells or particles. In this process, a sample containing cells or particles is suspended in a fluid and injected into the flow cytometer instrument. The sample is focused to ideally flow one cell at a time through a laser beam, where the light scattered is characteristic to the cells and their components. Cells are often labeled with antibodies coupled to fluorescent markers from which light is absorbed and then emitted in a band of wavelengths. Tens of thousands of cells can be quickly examined, and the data gathered are processed by a computer. Various bioinformatic approaches as outlined in the application can then been applied to the data. This includes high-dimensional viSNE and spanning-tree progression analysis for density-normalized events (SPADE) (52) as performed on Cytobank (cytobank.org).

As used herein, the term “Western blotting” refers to an analytical technique used to detect specific proteins in a sample of tissue homogenate or extract. The western blot technique uses three elements to achieve its task of separating a specific protein from a complex: separation by size, transfer of protein to a solid support, and marking the target protein using a primary and secondary antibody to visualize.

As used herein, the term “2-D gel electrophoresis” refers to a technique used to analyze proteins. This technique begins with electrophoresis in the first dimension and then separates the molecules perpendicularly from the first to create an electropherogram in the second dimension. The two dimensions that proteins are separated into using this technique can be isoelectric point, protein complex mass in the native state, or protein mass.

As used herein, the term “immunoassays” refers to a biochemical assay that measures the presence or concentration of a macromolecule or a small molecule in a solution through the use of an antibody or an antigen.

As used herein, the term “fluorescence-activated cell sorting” (FACS) refers to a technique that utilizes flow cytometry to separate cells based on morphological parameters and the expression of multiple extracellular and intracellular proteins. This method allows multiparameter cell sorting and involves encapsulating cells into small liquid droplets which are selectively given electric charges and sorted by an external electric field.

As used herein, the term “administer” or “administration” or “administering” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of routes of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmucosal, and inhalation.

An agent of the present disclosure can be administered for therapy by any suitable route of administration. It will also be appreciated that the optimal route will vary with the condition and age of the recipient, and the disease being treated.

As used herein, “therapeutically effective amount” of a drug or an agent refers to an amount of the drug or the agent that is sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose and may occur only after administration of a series of doses. Thus, a therapeutically effective amount may be administered in one or more administrations.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of the two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent more than 90%, often more than 95%, of all macromolecular species present in a purified preparation prior to admixture with other formulation ingredients. In other cases, the purified preparation may be essentially homogeneous, wherein other macromolecular species are not detectable by conventional techniques.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology,

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

The term “culturing” refers to growing cells in a culture medium under conditions that favor expansion and proliferation of the cell. The term “culture medium” or “medium” is recognized in the art and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for culture is a medium. Similarly, a powder mixture that when mixed with water or other liquid becomes suitable for cell culture may be termed a “powdered medium.” “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high-density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the disclosure while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

As used herein, the phrase “anti-inflammatory glucocorticoid” refers to any class of steroid hormones that bind to glucocorticoid receptors and reduce inflammation. Examples of anti-inflammatory glucocorticoids include cortisol (physiological glucocorticoids) and alclometasone, betamethasone, budesonide, ciclesonide, clobetasol, clocortolone, deprodone, desonide, dexamethasone, difluprenate, flunisolide, fluocinolone, fluticasone, halcinonide, halomethasone, halopredone, hydrocortisone, loteprednol, methylprednisolone, mometasone, naflocort, paramethasone, prednicarbate, prednisolone, prednisone, triamcinolone, rimexolone, and terbutaline, but are not limited thereto. In a specific embodiment, the anti-inflammatory glucocorticoid is dexamethasone, or a salt thereof. In some embodiments, dexamethasone has the following chemical formula:

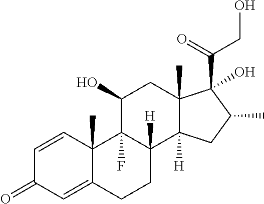

As used herein, the phrase “cross-sectional studies” refers to studies conducted at a single moment, while the phrase “longitudinal studies” refers to studies conducted over time. Cross-sectional studies provide a picture of the state of affairs, while longitudinal studies give an extended investigation of the problem. Longitudinal studies evaluate multiple measures over an extended period to detect trends and changes. While longitudinal studies repeatedly observe the same participants over a period of time, cross-sectional studies examine different samples of the population at one point in time.

As used herein, the term “an equivalent thereof” in reference to a polynucleotide or a protein (e.g., a capsid or coat protein) include a polynucleotide or a protein that comprise, or consists essentially of, or yet further consists of, at least 80%, 85%, 90%, 95%, 96%, 97%, 98%, or 99% sequence identify to the respective polynucleotide or protein of which it is compared to, while still retaining a functional activity. In the instances with reference to a capsid or coat protein, a functional activity refers to the formation of a virus or VLP.

### MODES FOR CARRYING OUT THE DISCLOSURE

To assess various markers that distinguishes severe COVID-19 disease, Applicant studied adults that were hospitalized at the McGill University Health Centre (MUHC) and confirmed by polymerase chain reaction (PCR) to be infected with SARS-CoV-2 [April 2020 and March 2021]. The median time from patient admission to the collection of cross-sectional samples was 3 days (IQR 1-8 days). For longitudinal studies, serial PBMC collections were obtained from 18 individuals at time points out to 36 days from admission. Flow cytometry analysis was conducted on 125 peripheral blood mononuclear cell (PBMC) collections (108 PBMC collections from 76 unique patients and 17 PBMC collections from healthy donors (HD)).

From the studies disclosed herein, Applicant provides methods of determining whether a subject suffering from an acute respiratory syndrome coronavirus 2 (SARS-Cov-2 or any variant thereof) or another human coronavirus (e.g., 229E, NL63, OC43, HKU1, MERS-CoV, or SARS-CoV) infection is more likely suffer from severe or mild disease, the methods comprising, or consisting essentially of, or yet further consisting of, detecting the amount of transcription factor TCF1+ (T-cell factor-1) expressing T cells in a biological sample comprising T cells that was isolated from the subject and comparing the measured amount to a reference amount, wherein a modified measured amount compared to the reference amount is indicative that the subject will suffer from severe or mild SARS disease. Any method to detect such can be used, several of which are disclosed herein. The T-cell is selected from a CD4+ or a CD8+ T cell.

In some embodiments, the method further comprises detecting the amount of LEF1 and/or Ki67 in the biological sample.

Another aspect of the disclosure is directed to methods for monitoring the progression of disease in a subject suffering from an acute respiratory syndrome coronavirus 2 (SARS-Cov-2 or any variant thereof) or another human coronavirus (e.g., 229E, NL63, OC43, HKU1, MERS-CoV, or SARS-CoV) infection, the methods comprising, or consisting essentially of, or yet further consisting of, detecting the amount of transcription factor TCF1+ (T-cell factor-1) expressing T cells in a biological sample comprising T cells that was isolated from the subject and comparing the measured amount to a reference amount over time, wherein a modified measured amount compared to the reference amount is indicative of the status of the SARS disease. Any method to detect such can be used, several of which are disclosed herein. The T-cell is selected from a CD4+ or a CD8+ T cell. In some embodiments, the method further comprises detecting the amount of LEF1 and/or Ki67 in the biological sample. In some embodiments, the monitoring comprises taking samples from the patient every hour, every three hours, every six hours, every twelve hours, every day, every three days, or once a week; measuring the markers (TCF1, LEF1 and/or Ki67) in CD4+ or CD8+ T cells.

As used herein, a “reference amount” refers to the measured amount of markers (e.g., TCF1, LEF1 and/or Ki67) from a healthy person (e.g., someone not infected with SARS-Cov-2 or another human coronavirus) or an average of measured amount of markers (e.g., TCF1, LEF1 and/or Ki67) from a healthy population. The reference amount may also refer to the measured amount of markers (e.g., TCF1, LEF1 and/or Ki67) from a sample from a subject taken before the subject before SARS-Cov-2 or another human coronavirus infection occurred.

In one aspect, the modified measured amount is an increase in the measured amount. In another aspect, the modified measured amount is a decrease in the measured amount. In a yet further aspect, the increase in the measured amount is indicative that the subject is more likely to suffer from mild disease.

In some embodiments, an increase in the measured amount of TCF1 is indicative that the subject is more likely to suffer from mild disease. In some embodiments, an increase in the measured amount of LEF1 is indicative that the subject is more likely to suffer from mild disease. In some embodiments, an increase in the measured amount of KI67 is indicative that the subject is more likely to suffer from mild disease. In some embodiments, an increase in the measured amount of TCF1, LEF1, KI67, or any combination thereof is indicative that the subject is more likely to suffer from mild disease. In some embodiments, an increase in the measured amount of TCF1, LEF1, and KI67 is indicative that the subject is more likely to suffer from mild disease.

In some embodiments, a decrease in the measured amount of TCF1 is indicative that the subject is more likely to suffer from severe disease. In some embodiments, a decrease in the measured amount of LEF1 is indicative that the subject is more likely to suffer from severe disease. In some embodiments, a decrease in the measured amount of KI67 is indicative that the subject is more likely to suffer from severe disease. In some embodiments, a decrease in the measured amount of TCF1, LEF1, KI67, or any combination thereof is indicative that the subject is more likely to suffer from severe disease. In some embodiments, a decrease in the measured amount of TCF1, LEF1, and KI67 is indicative that the subject is more likely to suffer from severe disease. In some embodiments, a loss of expression of TCF1, LEF1, KI67, or any combination thereof (i.e., a measured amount of about zero) is indicative that the subject is more likely to suffer from severe disease.

In some embodiments, an increase in the measured amount of TCF1, LEF1, KI67, or any combination thereof is indicative that the subject is more likely to suffer from long-term COVID-19. In some embodiments, an increase in the measured amount TCF1, LEF1, and KI67 is indicative that the subject is more likely to suffer from long-term COVID-19.

As used herein, the phrase “long-term COVID-19” refers to COVID-19 symptoms that continue for more than 12 weeks which cannot be explained by another cause. In adults, long-term COVID-19 symptoms include fatigue, sleep disturbances, shortness of breath, and general pain and discomfort. In some embodiments, adult long-term COVID-19 symptoms further include more severe cognitive issues such as memory loss, difficulty with concentration or thinking, as well as anxiety and depression. In children, long-term COVID-19 symptoms include symptoms such as fatigue, headaches, abdominal pain, sleep problems, muscle aches, joint pains and shortness of breath. In some embodiments, pediatric long term COVID-19 symptoms include long term cognitive problems, such as lack of concentration, difficulty thinking or concentrating.

In another aspect, the decrease in the measured amount compared to the reference amount is indicative that the subject is more likely to suffer from severe disease. Severe SARS disease can have a World Health Organization (WHO) severity score of 5 or higher. Alternatively, mild SARS disease has a World Health Organization (WHO) severity score of 4 or less.

In one aspect, the patient is clinically lymphopenic.

A non-limiting example of the biological sample comprises peripheral blood lymphocytes (PBMCs).

In one embodiment of this disclosure, the methods further comprise, or consist essentially of, or yet further consist of isolating the biological sample taken from the subject prior to the comparison. So, the method can include the further step of isolating or taking a further biological sample from the subject after treatment to the detecting step.

For those determined to be susceptible to severe illness, the method further comprises administering a therapy to treat severe SARS. For those determined not to be susceptible to severe illness, a therapy to treat mild to moderate SARS is administered. For patients or subjects susceptible to severe SARS, non-limiting examples of the therapy is selected from an anti-IL 12 therapy or an antibody cocktail therapy, e.g., an anti-IL-12 antibody therapy. Non-limiting examples of such include ustekinumab (STELARA®).

Alternatively or in combination, the therapy comprises an anti-inflammatory glucocorticoid, such as dexamethasone.

This disclosure also provides methods for treating acute respiratory syndrome coronavirus 2 (SARS-Cov-2 or any variant thereof) or another human coronavirus (e.g., 229E, NL63, OC43, HKU1, MERS-CoV, or SARS-CoV) infection and having a reduced amount of transcription factor TCF1+ (T-cell factor-1) expressing T cells comprising, or consisting essentially of, or yet further consisting of administering an effective amount of a therapy to treat severe SARS disease. In one aspect, the reduced amount of TCF1+ T cells is determined by a method comprising measuring and comparing the amount of TCF+ T cells in a biological sample isolated from the patient. The T-cell can be a CD4+ T cell or a CD8+ T cell.

Severe SARS disease can have a World Health Organization (WHO) severity score of 5 or higher. Alternatively, mild SARS disease has a World Health Organization (WHO) severity score of 4 or less. Mild SARS can have a WHO severity score of 4 or less. In one aspect, the patient is clinically lymphopenic.

A non-limiting example of the biological sample comprises peripheral blood lymphocytes (PBMCs).

The methods can further comprise, or consist essentially of, or yet further consist of isolating a biological sample from the subject prior to the comparison step. Alternatively or in addition, the method further comprises taking a further biological sample from the subject after the treatment step.

Non-limiting examples of therapies to use in the disclosed methods comprise, or consist essentially of, or yet further consist of, administration of therapy selected from an anti-IL 12 therapy or an antibody cocktail therapy. In one aspect, the anti-IL-12 therapy comprises an anti-IL-12 antibody therapy. In another aspect, the anti-IL-12 antibody therapy comprises ustekinumab (STELARA®). In another embodiment, the therapy comprises an anti-inflammatory glucocorticoid. In a further aspect, the anti-inflammatory glucocorticoid comprises dexamethasone.

In some embodiments, therapies to use in the disclosed methods comprise, or consist essentially of, or yet further consist of administering an effective amount of an antiviral therapy. In some embodiments, the antiviral therapy is selected from ritonavir, nirmatrelvir, ritonavir and nirmatrelvir combination (PAXLOVID®), molnupiravir (LAGEVRIO®) or remdesivir (VEKLURY®).

In some embodiments, therapies to use in the disclosed methods comprise, or consist essentially of, or yet further consist of administering an effective amount of an immune modulator. In some embodiments, the immune modulator comprises a Janus Kinase (JAK) inhibitor. In a specific embodiment, the JAK inhibitor comprises, or consists essentially of, or yet further consists of, baricitinib (OLUMIANT®). In some embodiments, the immune modulator comprises, or consists essentially of, or yet further consists of, an interleukin receptor 1 antagonist (IL-1Ra). In some embodiments, the IL-1Ra comprises anakinra (KINERET®). In some embodiments, the immune modulator comprises an antibody. In some embodiments, the antibody is an interleukin-6 (IL-6) receptor inhibitor. In a specific embodiment, the IL-6 receptor inhibitor antibody is Tocilizumab (ACTEMRA®). In some embodiments, the antibody is a human c5a antibody. In some embodiments, the human c5a antibody comprises vilobelimab (GOHIBIC®).

In some embodiments, therapies to use in the disclosed methods comprise, or consist essentially of, or yet further consist of administering an effective amount of at least one SARS-Cov-2-targeting antibody. In some embodiments, the SARS-Cov-2-targeting antibody comprises one or more of REGEN-COV® (casirivimab and imdevimab), sotrovimab, bamlanivimab and etesevimab, Bebtelovimabb, and EVUSHELD® (tixagevimab co-packaged with cilgavimab).

In a yet further aspect, the treatment is co-administered with an effective amount of a second therapy, that can be administered prior to, concurrently or subsequently to the treatment of the disclosed methods. The second therapy can be a different therapy or a different dose of the same primary therapy.

The method of this disclosure is for the treatment of any subject susceptible to a SARs-type infection, e.g., a mammal, such as a canine, a feline, an equine, a bovine, an ovine, a murine, a rat, a simian or a human patient. The gender of the subject is female or male and can be of any age, e.g., pediatric, adult or geriatric.

As noted above, any appropriate method for detecting can be used, non-limiting examples of such include a method comprising one or more of DNA microarrays, Real-time PCR, Chromatin immunoprecipitation (ChIP), flow cytometry, Western blotting, 2-D gel electrophoresis, immunoassays, or Fluorescence-activated cell sorting.

This disclosure also provides kits containing reagents to detect or measure the presence or amount of TCF1, Ki67 and/or LEF1 expressing T cells in a biological sample and instructions for use. In a further aspect, an effective amount of a suitable therapy is provided.

Clinical metadata was available from the COVID-19 patients over the course of their hospitalization (Table 1). The cohort studied had a median age of 70.3 years (IQR 59.5-81.7) and was predominantly male (74%). The time covered corresponded to the first and second waves of the COVID-19 pandemic in Montreal, Quebec, Canada which was marked by institutional spread (wave 1), followed by community exposure (wave 2) that preceded the availability of vaccines. The most commonly documented comorbidities in Applicant's cohort included hypertension (61%), respiratory disease (38%), cardiovascular disease (34%), diabetes and/or metabolic syndrome (32%), dementia (14%), immunosuppression (13%), active malignancy (11%) and chronic renal disease (9%). Presenting symptoms included fever (55%) and/or respiratory (71%), neurological (42%), gastrointestinal (37%) and cardiovascular (14%) compromise. In-hospital medications included antibiotics (86%), anticoagulants (78%), glucocorticoids (33%), antivirals (12%) and immunomodulators (5%). 28% of patients received advanced organ support (invasive mechanical ventilation (28%), parenteral vasopressors (22%), extracorporeal membrane oxygenation (ECMO) (8%) and/or renal replacement therapy (3%)).

TABLE 1
Demographics, Comorbidities, Clinical Presentation, Treatment & Outcomes

| | | Total | | Mild group | | Severe | | |
|---|---|---|---|---|---|---|---|---|
| | | (n = 76) | | (n = 43) | | (n = 33) | | p-value |
| Age (median) | | 70.3 | (59.5-81.7) | 72.9 | (58.8-82.3) | 68.9 | (62.7-79) | 0.888 |
| Sex (count) | Female | 20 | (26%) | 13 | (30%) | 7 | (21%) | 0.376 |
| | Male | 56 | (74%) | 30 | (70%) | 26 | (79%) | |
| Wave (count) | 1 | 32 | (42%) | 17 | (40%) | 15 | (45%) | 0.604 |
| | 2 | 44 | (58%) | 26 | (60%) | 18 | (55%) | |
| BMI (median)* | | 27.5 | (23.7-31.9) | 27.0 | (23.7-31.9) | 28.5 | (23.4-33.4) | 0.643 |
| Provenence | Home | 58 | (76%) | 35 | (81%) | 23 | (70%) | 0.312 |
| (count) | Assisted living | 9 | (12%) | 3 | (7%) | 6 | (18%) | |
| | residence | | | | | | | |
| | Other institution | 9 | (12%) | 5 | (12%) | 4 | (12%) | |
| | (nosocomial) | | | | | | | |
| Comorbidities | Cardiovascular | 26 | (34%) | 17 | (40%) | 9 | (27%) | 0.264 |
| (count) | Disease | | | | | | | |
| | Hypertension | 46 | (61%) | 29 | (67%) | 17 | (52%) | 0.159 |
| | Dislipidemia | 25 | (33%) | 16 | (37%) | 9 | (27%) | 0.361 |
| | Renal Disease | 7 | (9%) | 4 | (9%) | 3 | (9%) | 1.000 |
| | Respiratory Disease | 29 | (38%) | 15 | (35%) | 14 | (42%) | 0.502 |
| | Immunsuppression | 10 | (13%) | 7 | (16%) | 3 | (9%) | 0.499 |
| | Active Malignancy | 8 | (11%) | 4 | (9%) | 4 | (12%) | 0.722 |
| | Diabetes | 24 | (32%) | 18 | (42%) | 6 | (18%) | 0.028 |
| | Dementia | 11 | (14%) | 5 | (12%) | 6 | (18%) | 0.421 |
| Presenting | Gastrointestinal | 29 | (38%) | 20 | (47%) | 9 | (27%) | 0.087 |
| Symptoms | Respiratory | 54 | (71%) | 30 | (70%) | 24 | (73%) | 0.888 |
| (count) | Fever | 42 | (55%) | 25 | (58%) | 17 | (52%) | 0.565 |
| | Neurological | 32 | (42%) | 23 | (53%) | 9 | (27%) | 0.022 |
| | Cardiovascular | 11 | (14%) | 9 | (21%) | 2 | (6%) | 0.101 |
| Treatment | antibiotics | 65 | (86%) | 38 | (88%) | 27 | (82%) | 0.421 |
| (count) | antivirals | 9 | (12%) | 2 | (5%) | 7 | (21%) | 0.036 |
| | glucocorticoids | 25 | (33%) | 11 | (26%) | 14 | (42%) | 0.121 |
| | immunomodulators | 4 | (5%) | 0 | (0%) | 4 | (12%) | 0.032 |
| | anticoagulants | 59 | (78%) | 35 | (81%) | 24 | (73%) | 0.369 |
| | Advanced organ | 21 | (28%) | 0 | | 21 | (64%) | 0.000 |
| | support | | | | | | | |
| | invasive mechanical | 21 | (28%) | 0 | | 21 | (64%) | |
| | ventailation | | | | | | | |
| | ECMO | 6 | (8%) | 0 | | 6 | (18%) | 0.000 |
| | vasopressors | 17 | (22%) | 0 | | 17 | (52%) | 0.000 |
| | renal replacement | 2 | (3%) | 0 | | 2 | (6%) | 0.000 |
| | therapy | | | | | | | |
| Outcomes | WHO score max | 4 | (3-7) | 3 | (3-4) | 7 | (7-8) | 0.000 |
| | (median) | | | | | | | |
| | Length of | 13 | (7-32) | 10 | (6-13) | 30 | (18-63) | 0.000 |
| | Hospitalization | | | | | | | |
| | (median) | | | | | | | |
| | Disposition (count) | | | | | | | 0.000 |
| | home | 47 | (62%) | 33 | (77%) | 14 | (42%) | |
| | institution | 16 | (21%) | 10 | (23%) | 6 | (18%) | |
| | Death in hospital | 13 | (17%) | 0 | (0%) | 13 | (39%) | |

The hospitalized patients were categorized clinically with either mild or severe disease as per their maximal severity scores using the World Health Organization (WHO)'s ordinal scale [47, 48]. Of the 51 individuals (67% of the total cohort) who were initially scored as having mild disease at the time of their initial PBMC collection, 8 (11%) ultimately progressed to severe disease of whom 4 (5%) died from disease complications, whereas 9 of 33 individuals who were initially scored as severe died of disease complications (Table 2).

TABLE 2
Clinical Lab Values of Covid patients

| | Total (n = 76) | | Mild group (n = 43) | | Severe (n = 33) | | p-value |
|---|---|---|---|---|---|---|---|
| O2 saturation (%) nadir | 90 | (88-92, | 92 | (90-93, | 88 | (83-90, | 0.000 |
| (median) | | n = 76) | | n = 43) | | n = 33) | |
| CRP (mg/L) (median) | 165 | (86-219, | 121 | (52-189, | 194 | (163-275, | 0.000 |
| | | n = 70) | | n = 37) | | n = 33) | |
| absolute lymphocyte count | 0.55 | (0.35-0.78, | 0.65 | (0.44-0.89, | 0.38 | (0.20-0.64, | 0.001 |
| nadir (×10⁹/L) (median) | | n = 76) | | n = 43) | | n = 33) | |
| ALT (U/L) (median) | 54 | (30-127, | 31 | (26-54, | 122 | (50-241, | 0.000 |
| | | n = 53) | | n = 25) | | n = 28) | |
| 25D (nM) (median) | 63 | (40-101, | 72 | (48-107, | 56 | (32-81, | 0.110 |
| | | n = 63) | | n = 37) | | n = 26) | |
| 25D deficient (<25 nM) | 9 | (14%, | 3 | (8%, | 6 | (23%, | 0.144 |
| (count) | | n = 63) | | n = 37) | | n = 26) | |
| 25D insufficient (<50 nM) | 22 | (35%, | 11 | (30%, | 11 | (42%, | 0.303 |
| (count) | | n = 63) | | n = 37) | | n = 26) | |
| 25D insufficient (<75 nM) | 36 | (57%, | 21 | (57%, | 15 | (58%, | 0.941 |
| (count) | | n = 63) | | n = 37) | | n = 26) | |

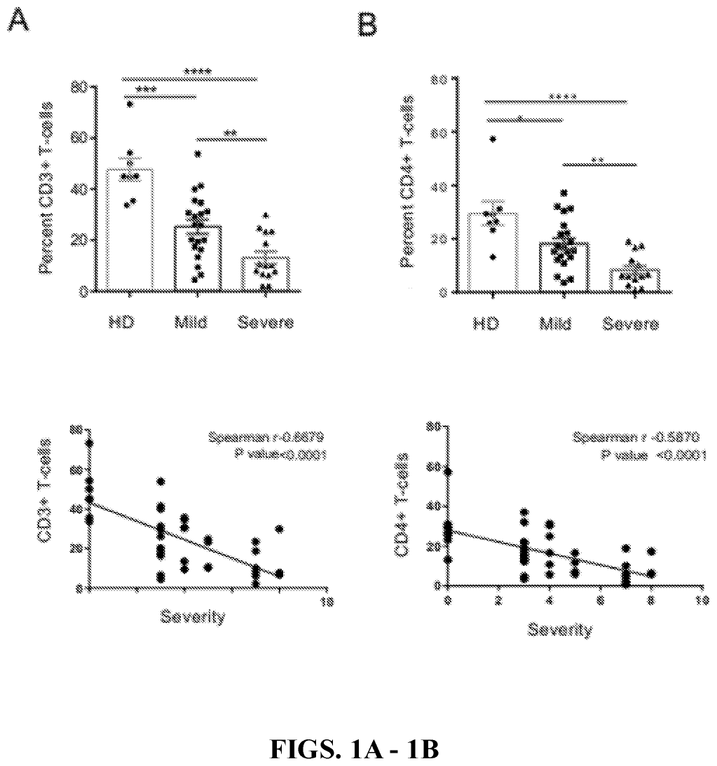

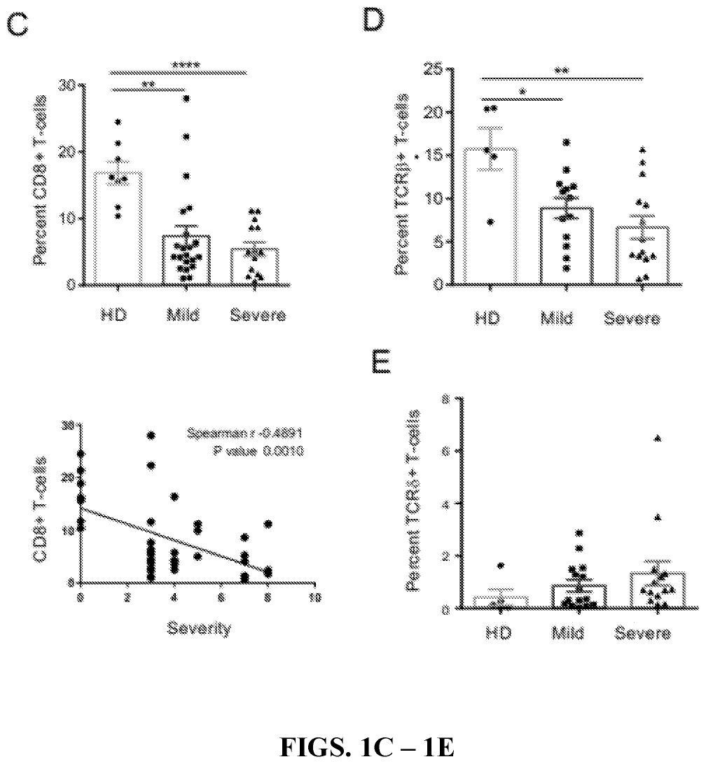

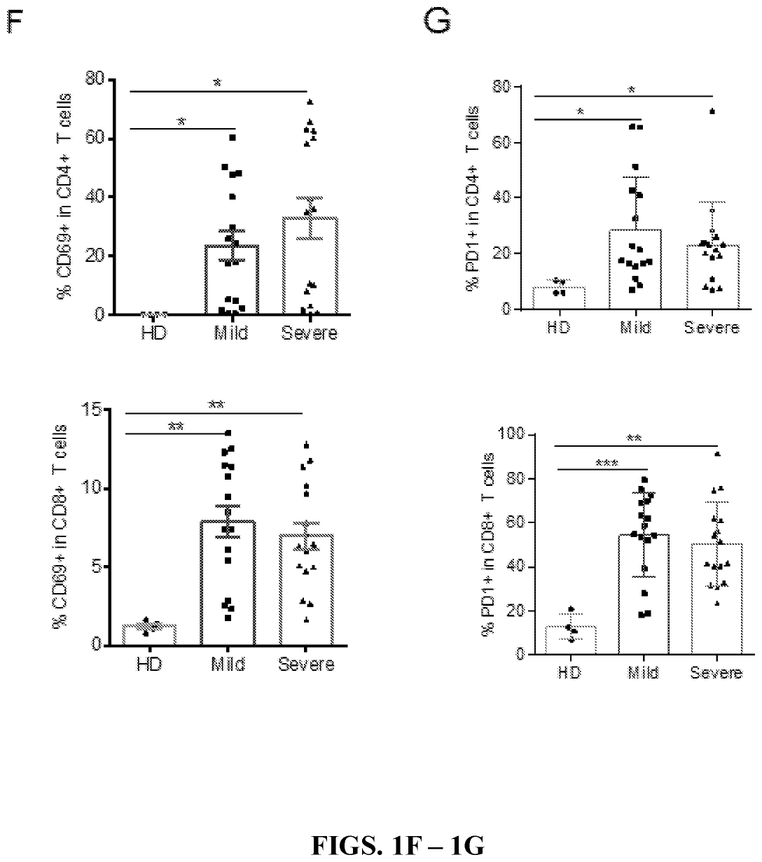

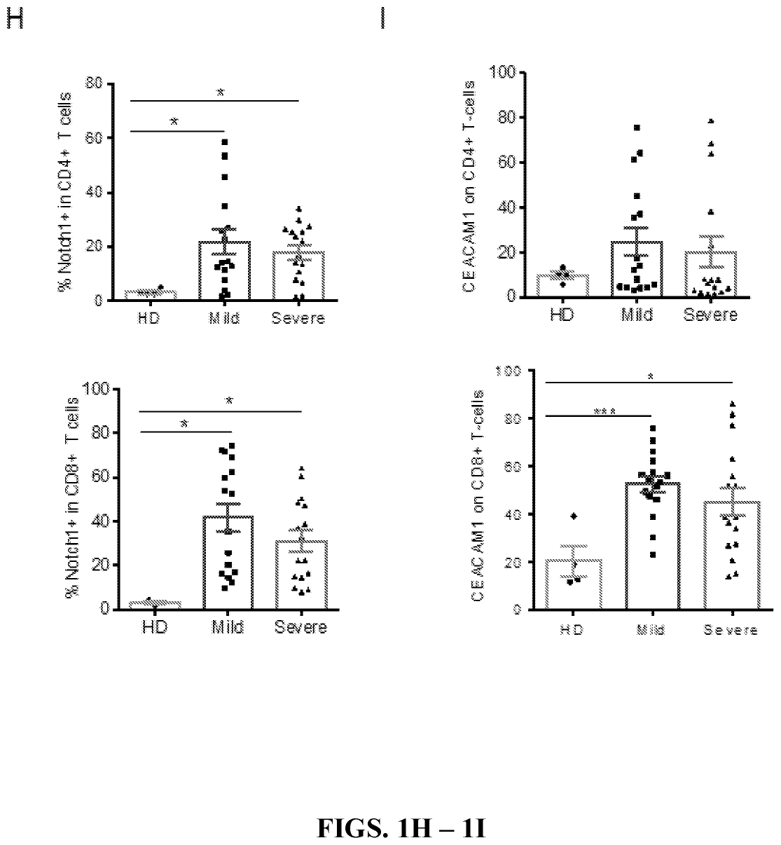

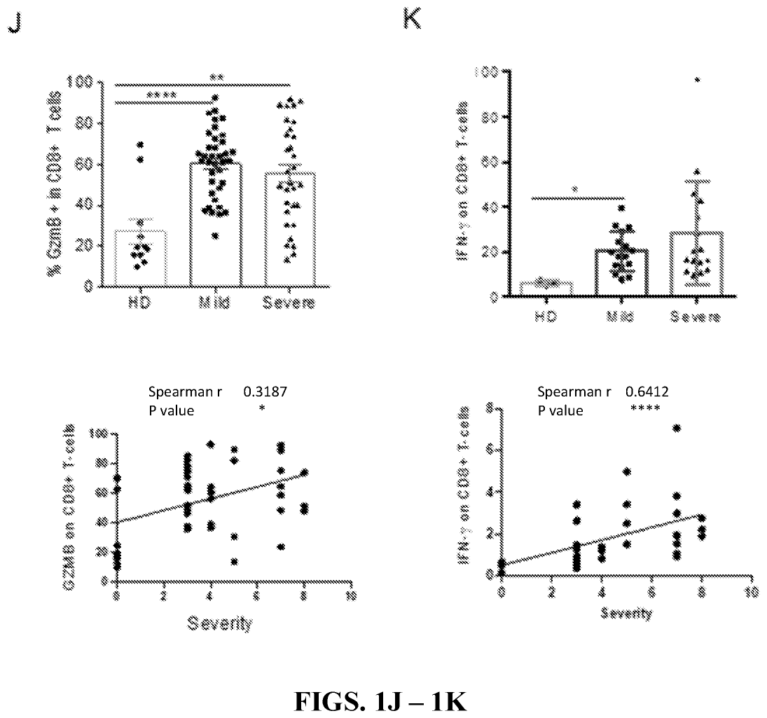

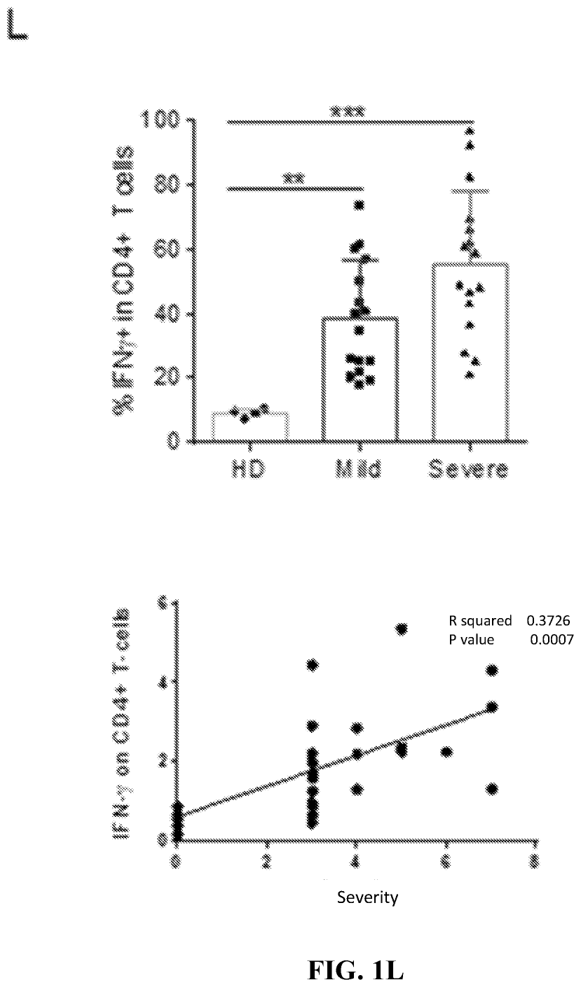

To initiate Applicant's investigation of the immune responses to mild and severe SARS-CoV-2 infection, Applicant compared the presence of T-cells from Applicant's patients with COVID-19. Overall, 66% of Applicant's hospitalized cohort were clinically lymphopenic by the time of their first peripheral blood mononuclear cell (PBMC) collection (absolute lymphocyte count <1000/mL) with 86% having developed lymphopenia at some point during their hospitalization. Applicant noted a progressive decline in the presence of CD3+ T-cells (FIG. 1A), CD3+CD4+ (FIG. 1B) and CD3+CD8+ peripheral T-cells (FIG. 1C) in comparing healthy donors to COVID-19 patients with mild or severe disease. This lymphopenia was confirmed by Spearman analysis which showed a progressive decline in CD3+, CD3+CD4+ and CD3+CD8+ T-cells with an increasing disease severity as defined by WOS (53, 54) (FIGS. 1A-C, lower panels). The staining of T-cells with antibody to the TCR-β+ also showed a decrease in cells (FIG. 1D), while staining of the smaller proportion of T-cells expressing TCR-γ+ failed to show a loss of cells (FIG. 1E). These findings are consistent with previous reports of lymphopenia during COVID-19 (22, 23, 55).

Applicant also noted the increase in the expression of activation receptors on CD8+ and CD4+ T-cells in samples from patients with mild and severe disease (FIGS. 1F-I, upper and lower panels, respectively), as reported (6, 13, 56, 57). As examples, this increase was observed for the activation antigen, CD69 (FIG. 1F), the inhibitory receptor (IR) PD-1 (FIG. 1G), and the differentiation antigen, Notch (FIG. 1H). A significant increase was seen for the IR, CEACAM1 on CD8+ T-cells (FIG. 1I, lower panel), while an increased trend was observed in CD4+ T-cells (FIG. 1I, upper panel). An increase in the effector granzyme B (GZMB) was seen in CD8 cells (FIG. 1I, upper panel), and was confirmed in relation to the WOS severity score (Spearman analysis: r=0.3, p=*) (lower panel). Further, an increase in the cytokine IFN γ1 was observed in CD8 and CD4 T-cells in mild and severe patients (FIG. 1K, FIG. 1L, upper panels, respectively) that was correlated with the WOS severity score (lower panels). Despite this, the increase was observed in samples from mild and severe disease but did not distinguish between these two groups in a statistically significant manner.

Preferential Loss of TCF1 Progenitor T-Cells in Patients with Severe Disease

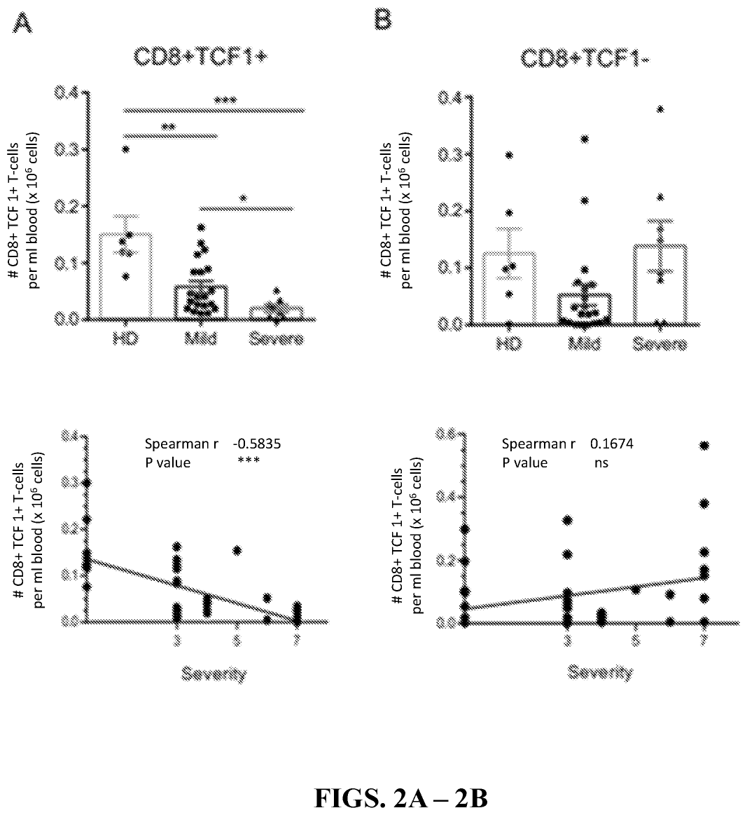

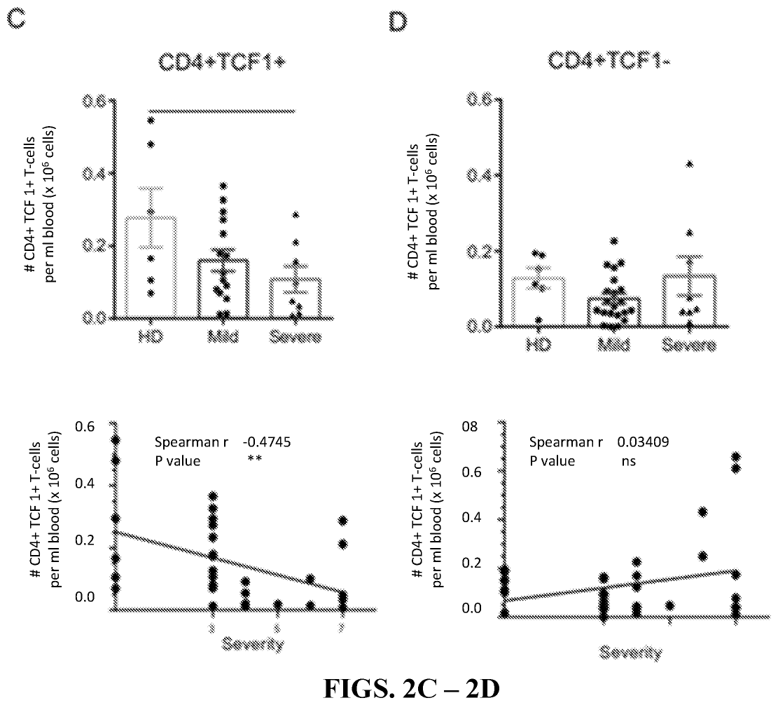

Given this, Applicant was next interested in identifying a specific molecule that could distinguish severe from mild disease. Applicant reasoned that progenitor T-cells might be affected given their central importance in the renewal of the peripheral T-cell compartment (34, 35). In this context, the transcription factor TCF-1 (Tcf7) defines a key subset of progenitor T-cells (28). To begin, Applicant simply assessed the numbers of TCF1+ versus TCF1− T-cells per ml of blood from healthy donors (HD) and patients with mild (M) or severe(S) disease (FIG. 2). Surprisingly, Applicant observed the preferential loss of CD8+TCF1+ cells in blood of severe patients when compared to healthy donors (HDs) or patients with mild disease (FIG. 2A, upper panel). A statistically significant difference was seen between HD and M samples as well as between M and S patients. Further, Spearman analysis confirmed a decrease in CD8+TCF1+ cells commensurate with increasing disease severity from a WOS ranking of 5-7 (r=−0.5835; p<0.005) (lower panel). By contrast, Applicant observed no reproducible change in numbers of CD8+TCF1− T-cells (FIG. 2B). In addition, Spearman analysis also failed to show a decrease in TCF1-cells commensurate with increasing disease severity from a ranking of 5-7 (lower panel). Applicant also noted a loss of CD4+ TCF1+ peripheral T-cells in patients with severe disease relative to healthy donors and patients with mild disease (FIG. 2C, upper panel). Further, Spearman analysis confirmed a decrease in CD4+ TCF1+ cells commensurate with increasing disease severity from a WOS ranking of 5-7 (FIG. 2C, lower panel). By contrast, Applicant observed no reproducible change in the presence of CD4+ TCF1− T-cells (FIG. 2D, upper panel) or by Spearman analysis (lower panel). These data indicated that there is a preferential loss of CD8+ and CD4+ TCF1+ T-cells in patients with severe versus mild COVID-19.

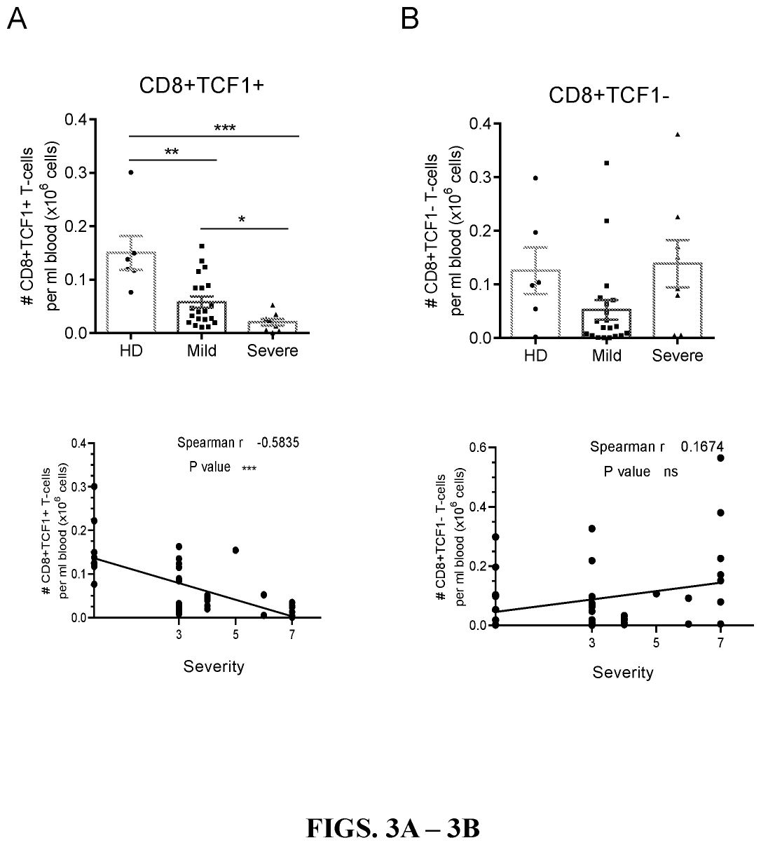

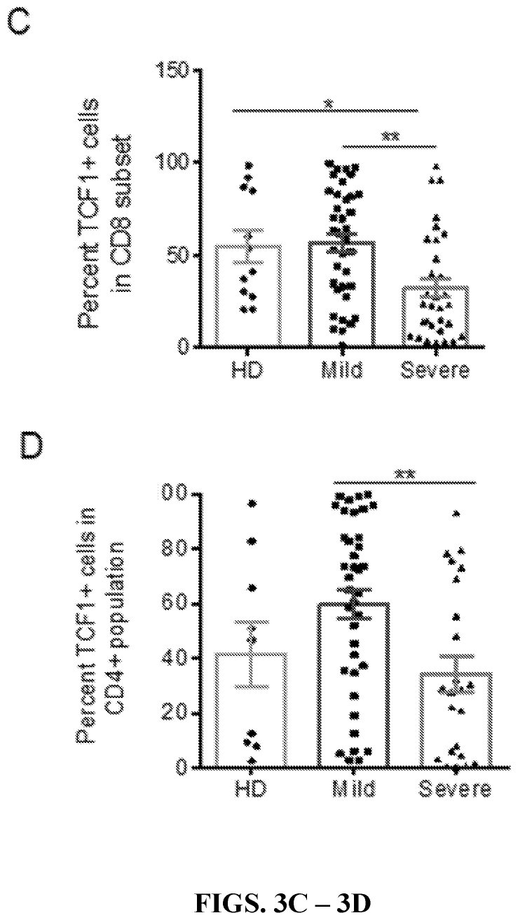

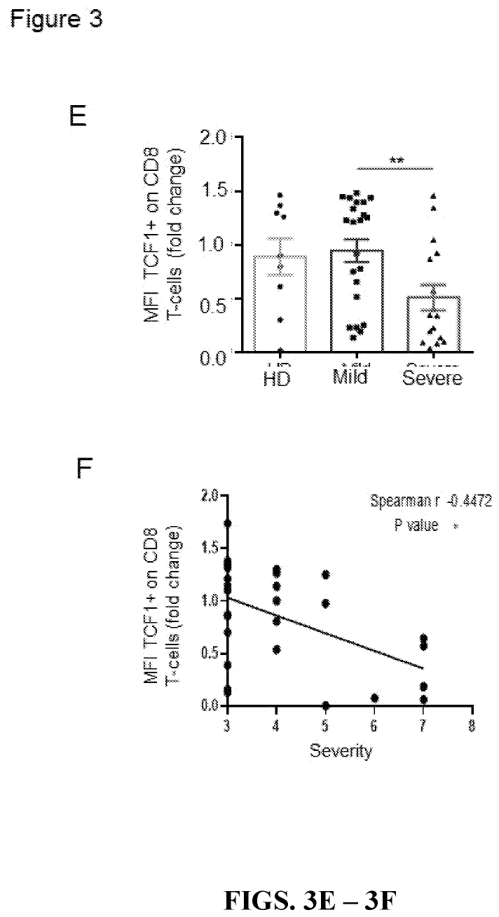

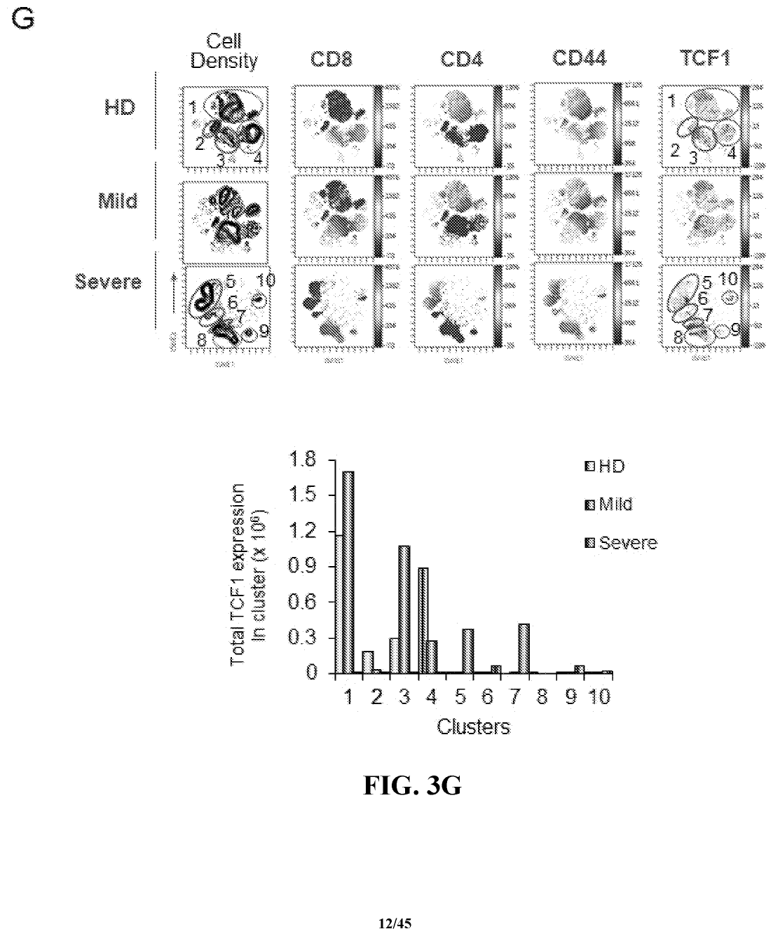

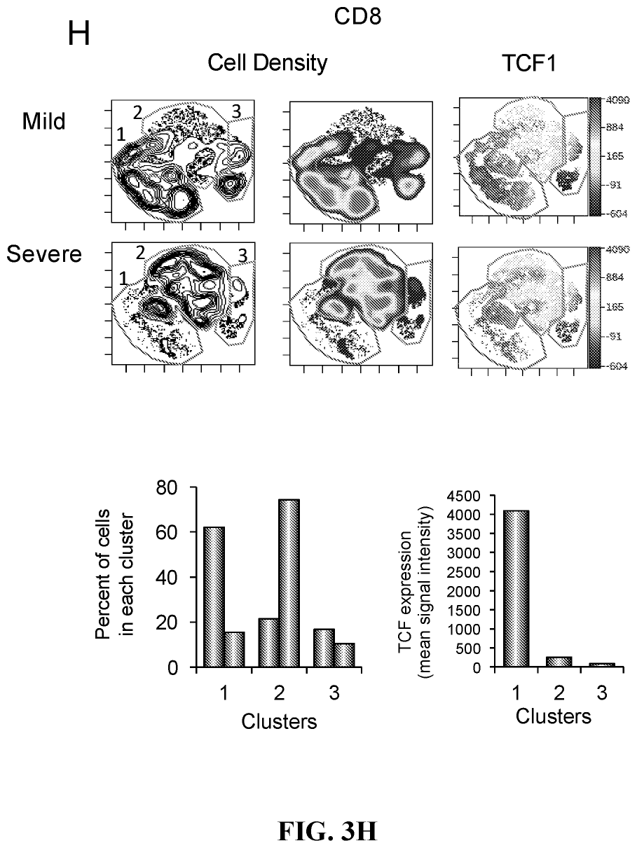

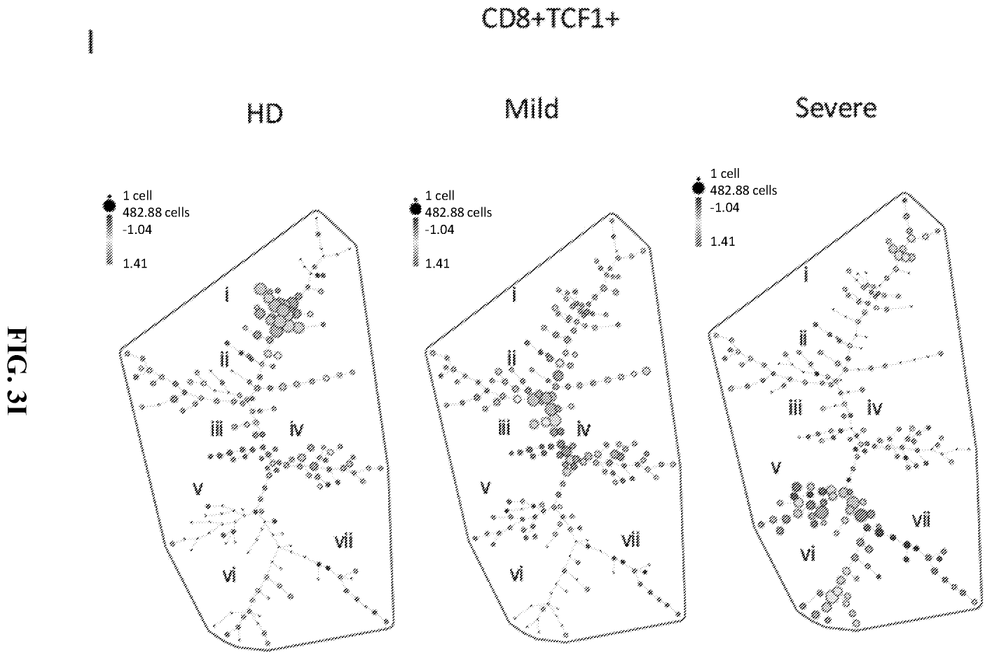

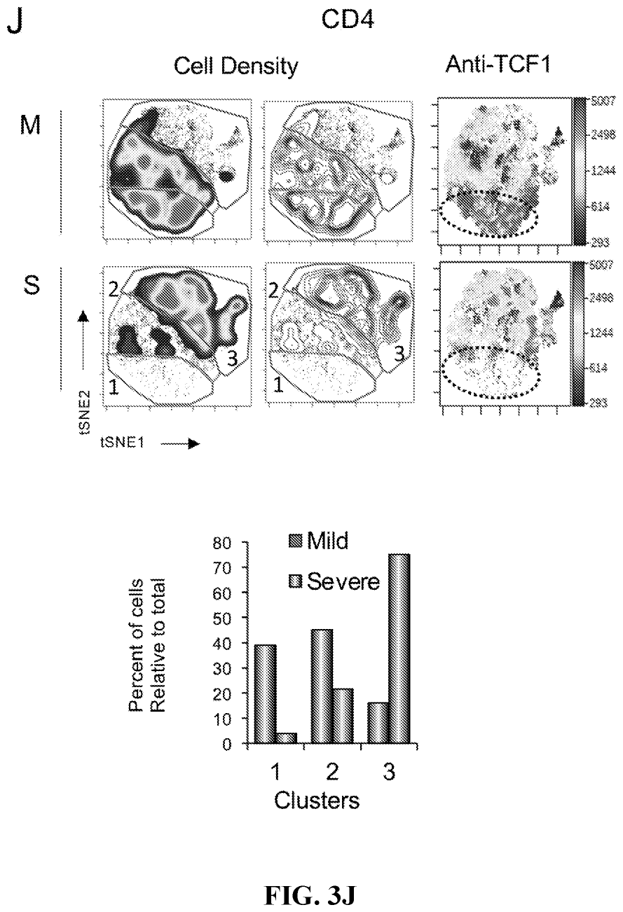

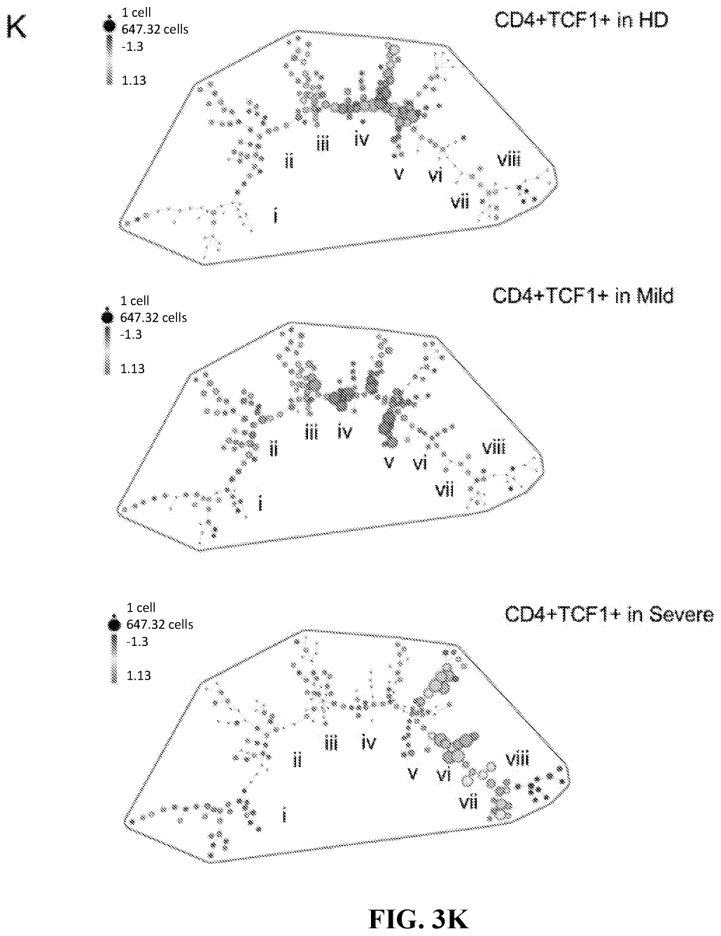

To examine this in more detail, Applicant repeated the presentation of the analysis for CD8+TCF1+ T-cells in peripheral blood from severe versus mild patients (FIG. 3A, B). Further, when samples were grouped together into mild (WOS 2-4) or severe (WOS 5-8), Applicant also noted a clear statistical decrease in % of CD8+TCF1+ (FIG. 3C) and CD4+ TCF1+ peripheral human T-cells between M and S samples (FIG. 3D). When expressed as an MFI value for TCF1+ expression in CD8+ T-cells, Applicant also observed a statistical reduction of TCF1 expression in the CD8+ subset (FIG. 3E). Spearman analysis also showed a decrease in TCF1 expression in CD8+ cells commensurate with disease severity (r=−0.47, p=*) (FIG. 3F). These data showed for the first time that TCF1+ CD8 and CD4+ T-cells were preferentially lost in the PBMCs from patients with severe disease. To Applicant's knowledge, this observation had not been reported in previous studies (6, 56, 58).

Next, Applicant examined the cellular populations using high-dimensional viSNE and spanning-tree progression analysis for density-normalized events (SPADE) (52). A representative viSNE densiometric profile showed multiple clusters or islands of cells (see upper and lower right panels) (FIG. 3G). Cell density profiles showed a similar positioning of clusters (1-4) for samples from HD and M patients. By contrast, samples from S patients showed a remarkable change in clustering patterns with the loss of clusters 1˜4 and the appearance of new clusters 5-10. CD3+CD4+ cells were found in clusters 1, 5, 10, CD3+CD8+ T-cells in clusters 3, 4, 7-9 and an intriguing population of CD3+CD4−CD8-T-cells in islands 2 and 6. Within clusters 5-10, Applicant observed that a marked lose in the expression of TCF1 (upper right panels; lower histogram). CD4+, CD8+ and CD4−CD8−cells showed a loss of TCF1. By contrast, the expression levels of CD8, CD4 and CD44 remained high in the various clusters.

A similar observation was made when Applicant gated solely on the CD8+ T-cells (FIG. 3H). This is shown for M and S patients where cell density analysis showed the presence of most cells in clusters 1 and 3 in M patients (upper left and middle panels). The reduction in the number of cells in these clusters in samples from severe patients was accompanied by a marked increase in the number of cells in cluster 2 (lower left and middle panels; also see lower left histogram). Cluster 2 showed a reduced overall level of TCF1 expression (upper and lower right panels; also lower right histogram). The same reduction in TCF1 was seen in SPADE (FIG. 3I). Here, Applicant concatenated samples from 9 patients in each treatment group involving multiple markers (12 different antibodies). Equal numbers of cells per treatment group were analyzed. SPADE allowed for better visualization of single-cell data involving down sampling, clustering and a minimum-spanning tree depiction of cellular heterogeneity. The fluorescence intensity of the different markers for each node is represented by color, while the node size represents the number of cells. From this, Applicant found that SPADE revealed a remarkable level of CD8 cell heterogeneity (>90 nodes of distinct CD8+ cells) with multiple tree branches depicting different cell groupings with the CD8+ population. Within each pattern, Applicant focused on 7 groupings of tree clusters and their nodes (i-vii). Within HDs, most of the cells were found in tree i with multiple nodes with high TCF1+ expression (left panel). In M CD8+ T-cells, Applicant observed a mild shift with increasing numbers of cells in tree groupings ii, iii and iv and loss of cells in tree i with moderate levels of TCF1 expression (middle panel). By contrast, the pattern in S patients underwent further regrouping with most cells in tree groupings v-vii (right panel). Although a small subset of nodes retained expression in tree v, the majority showed a decrease in TCF expression in tree groupings vi-vii (as seen by the colors light pink and blue). Some nodes showed a loss of TCF1 expression (vii). Both viSNE and SPADE analyses showed a reduction in the expression of TCF1 and the presence of TCF1+CD8+ cells in the transition from HD/mild to severe disease.

Similar findings were seen when Applicant gated solely on the CD4+ T-cells (FIG. 3J). viSNE profiles showed similar groupings of cells in clusters 1-3 for HD and M patients. TCF1 expression was similar if not higher in the samples from mild patients in cluster 3. By contrast, these clusters were mostly lost and replaced by clusters 4 and 5 with low TCF1 expression (lower panel; also see lower histogram). SPADE analysis showed a multiplicity of nodes for CD4+ cells (FIG. 3K). Within this complexity, Applicant separated 8 tree groupings (i-viii). Most of the TCF1+ cells were seen in grouping iii-v in HD CD4+ samples (upper image) and in samples from patients with mild disease (middle panel). The level of TCF1 expression even increased in patients with mild disease in nodes in tree groupings iv and v. However, by contrast, the pattern from severe patients showed a contraction in the presence of cells in tree groupings i-iv, instead, replaced by cells in tree groupings v-viii. Further, each of the nodes in v and vii showed a decrease in the expression of TCF1 as seen by lower intensity pink, green and blue colors (see side scale). These data showed that CD4+ cells, as with CD8+ T-cells showed a remarkable change in TCF1 expression in the transition from HD/mild to severe disease.

### The Loss of TCF1 Expression in T-Cell Subsets

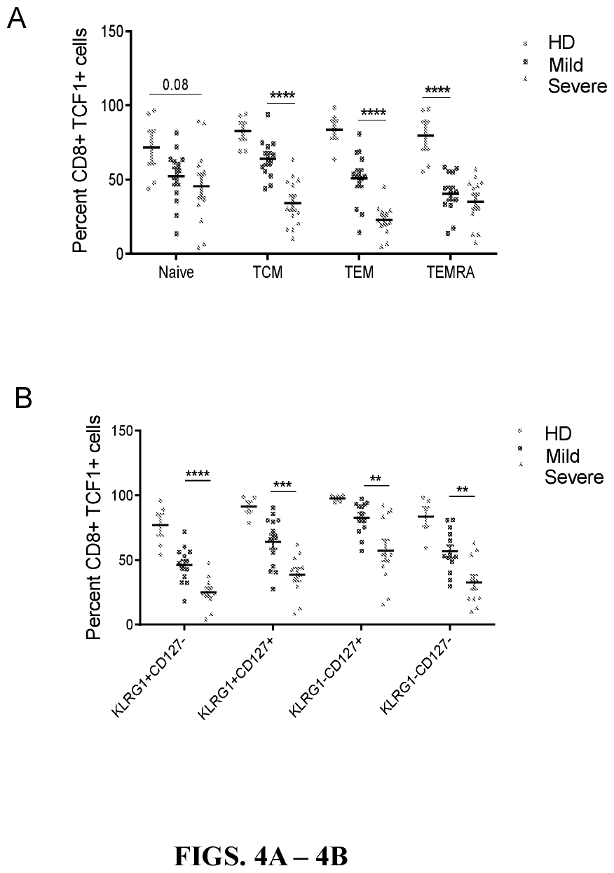

Applicant next examined TCF1 expression on different subsets of CD8+ T-cells. CD8+ can be divided into naïve, memory and effector-memory subsets (41, 59). TCF1 is expressed in naïve T-cells as well as differentiated T-cells which undergo some self-renewal (60) and are need for responses to anti-PD-1 blockade (39, 40). Human central memory (TCM, CCR7+CD45RA−), effector memory (TEM, CCR7−CD45RA−), and effector memory cells re-expressing CD45RA subset (TEMRA, CCR7−CD45RA+) were analyzed. The question was whether the presence of TCF1+ cells was reduced only in naïve cells, or whether the cells were lost amongst other subsets. In fact, the presence of TCF1+ cells was reduced in all subsets of T-cells of patients with severe and mild disease. (FIG. 4A). A trend was seen in naïve cells, while significant differences were seen in TCM and TEM in comparing mild vs severe disease. Surprisingly, TEMRAs showed a significant reduction in CD8+ T-cells in patients with both mild and severe disease. These data indicated that severe disease was accompanied by a major reduction in TCF1+ expression in CD8+ T-cells.

The receptors KLRG1 and CD127 can also define subsets of mature CD8+ T-cells. Killer cell lectin-like receptor G1 (KLRG-1) is a marker for highly cytotoxic and proliferative effector CD8+ T cells (61, 62). KLRG1⁺ effector CD8⁺ T cells lose KLRG1 and differentiate into all memory T cell lineages (59, 63). CD127 (IL-7 receptor) provides long lasting pro-survival signals (64). In this context, KLRG1+CD127-correspond to short lived effector T-cells (SLECs), while KLRG1^{lo} CD127+ cells correspond to memory precursor effector cells (MPECs) (59). Despite this, reductions in the presence of TCF1 were seen in the subsets defined by KLRG1+CD127−, KLRG1+CD127+, KLRG1−CD127+ and KLRG1−CD127− expression (FIG. 4B). Interestingly, the percent representation of TCF1+ cells was reduced in all subsets including the SLEC and MPEC subsets in cases of severe disease. These data showed that TCF1 expression was broadly reduced in multiple subsets of CD8+ cells from naïve cells to those in various stages of differentiation.

Reduced Expression of LEF1, Notch 1 in CD8+ T-Cells from Severe Patients

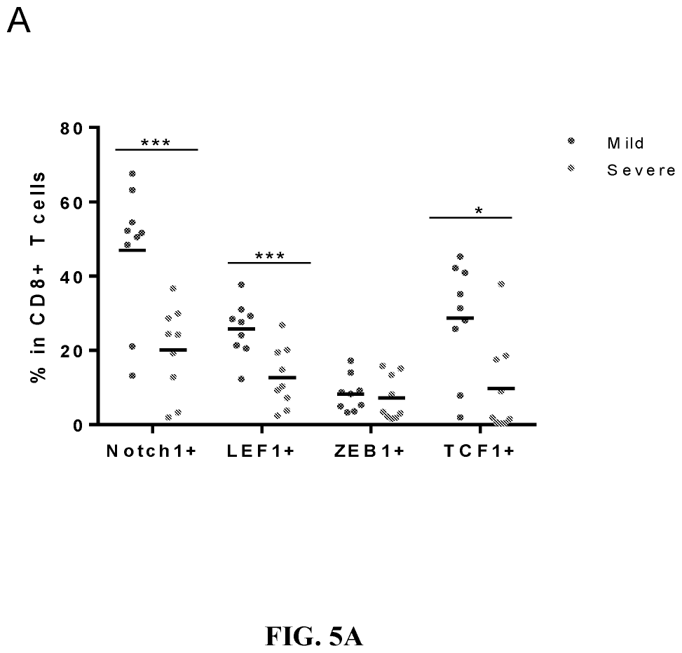

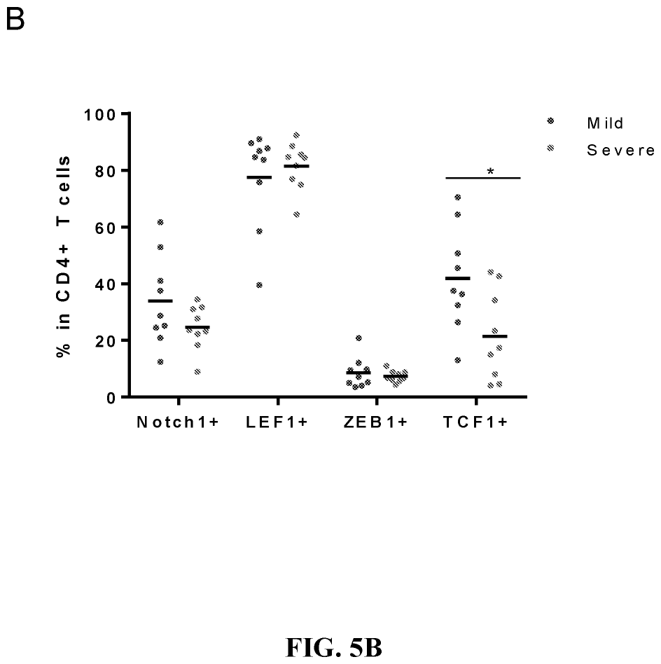

Applicant also reviewed the expression of other markers for progenitor cells such as LEF1 (for lymphoid enhancer-binding factor), Notch 1, ZEB1 (TCF8) and TCF1 in a smaller sample size (FIG. 5). LEF-1 (for lymphoid enhancer-binding factor) interacts with beta-catenin (45), while the combined effects of TCF1 and LEFI provide constant supervision of the differentiation states of CD8+ T-cells (46). Similarly, Notch 1 signaling is required for commitment of thymic T-cell progenitors, promoting proliferation and survival (47). In Applicant's hands, together with TCF1, Notch 1 and LEFI were able to distinguish severe from mild states within the CD8 subset (FIG. 5A). Reduced Notch 1 expression was also observed in CD4+ T-cells in patients with severe relative to mild disease (FIG. 5B). By contrast, as a control, the transcription factor ZEB showed no distinguishing effect in either the CD4 or CD8 compartments. ZEB represses T-lymphocyte-specific Interleukin 2 expression by binding to a negative regulatory domain next to the IL-2 transcription start site (48). These data, including the loss of LEF1 and Notch1+ cells in the CD8 compartment and Notch 1 in the CD4 compartment (together with TCF1) in distinguishing severe from mild disease underscored the importance of loss of progenitor T-cells in the development of severe COVID-19.

### The Loss of TCF1 Expression in Longitudinal Analysis of Patients

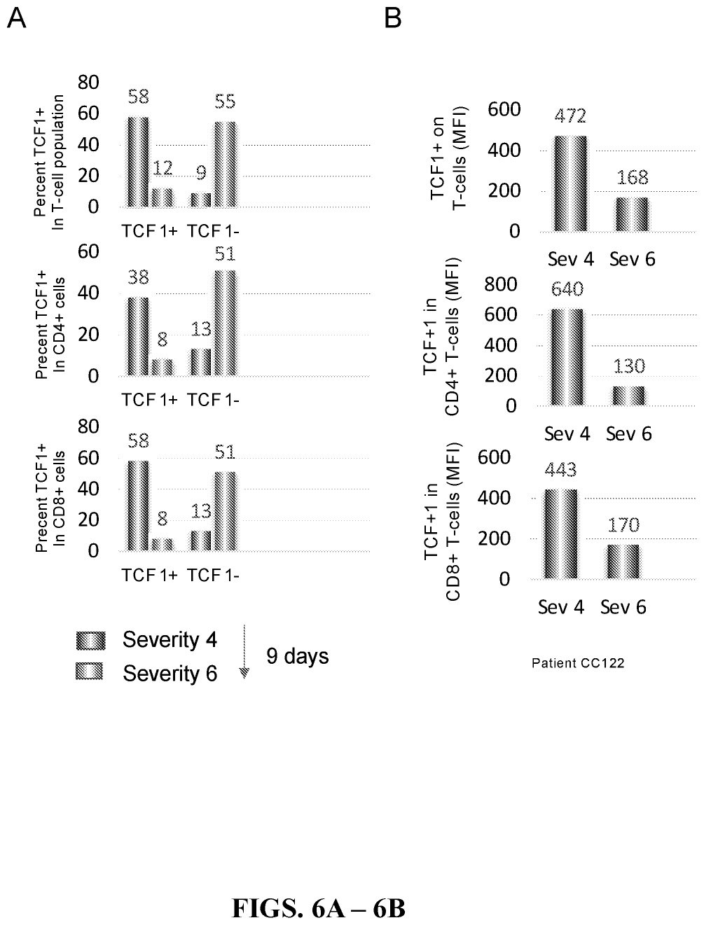

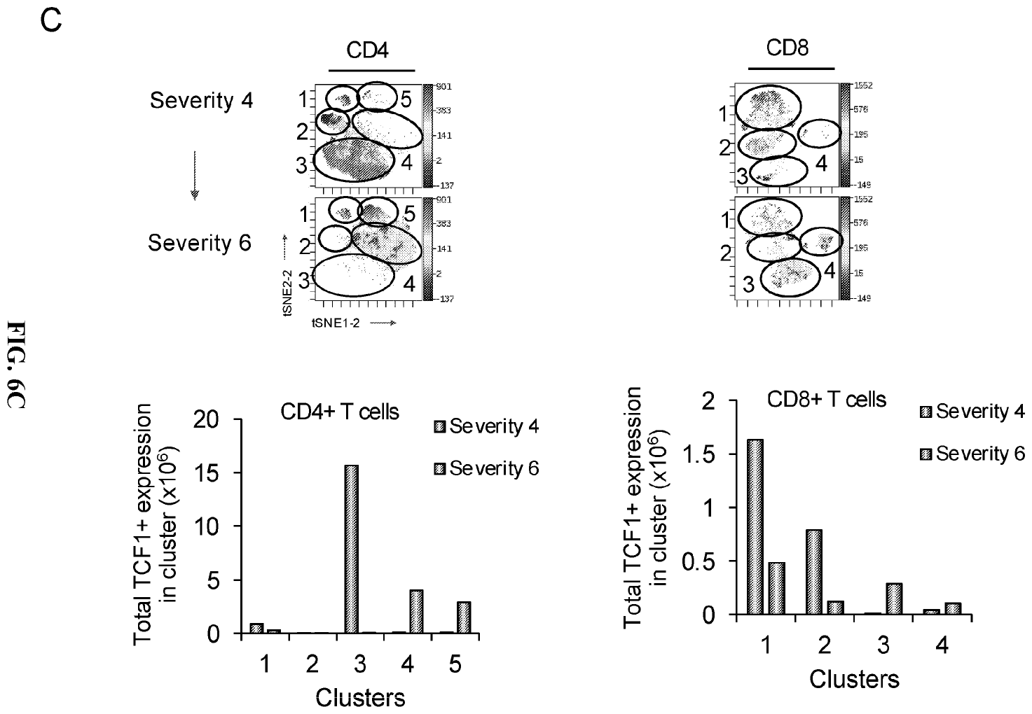

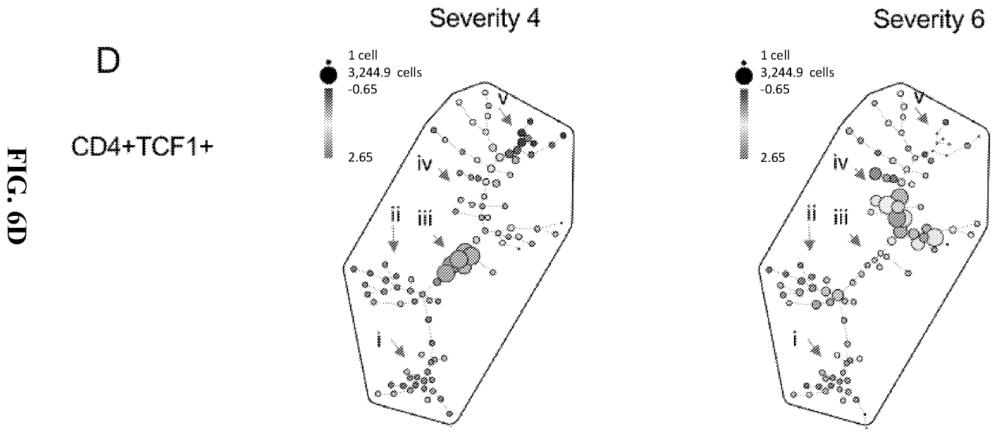

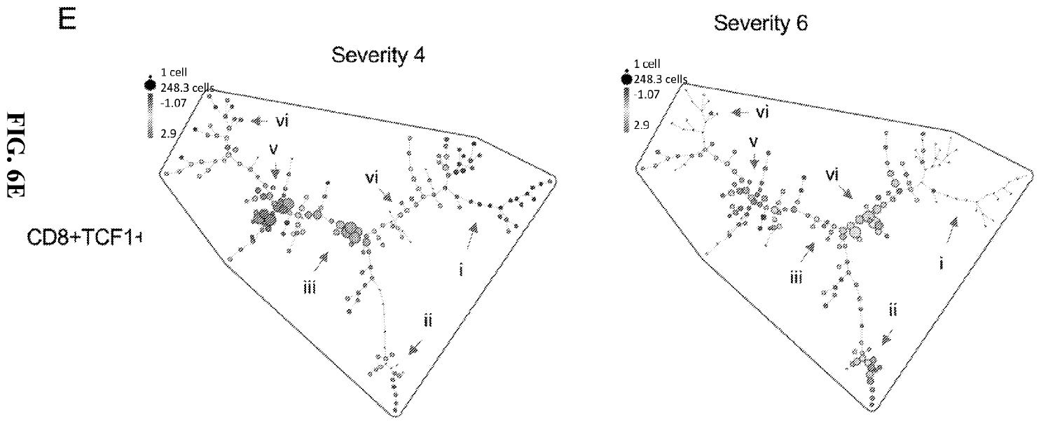

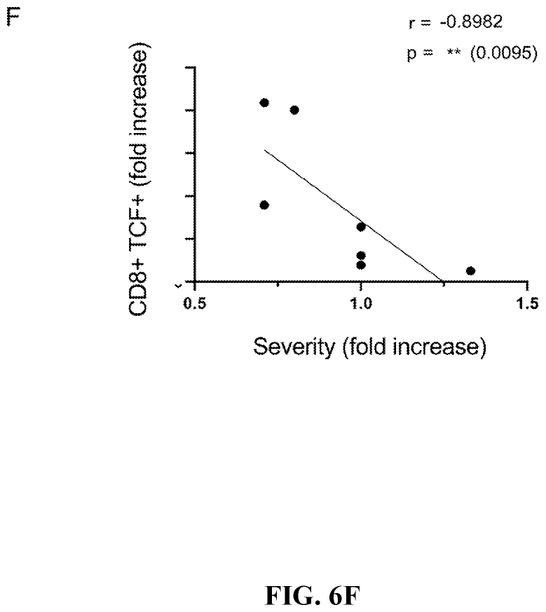

Applicant next carried out longitudinal analysis which showed a loss of TCF1+ CD4 and CD8 cells during progression to severe disease (FIG. 6). As a representative example, in the case of patient CC122, the initial sample was taken from the patient at a time when they had a severity ranking of 4. A second sample was taken 9 days later from the same patient who then had a severity ranking of 6. Applicant first observed a decrease in the percent of TCF1+ in the T-cell population from 58 to 12 percent (FIG. 6A, upper panel). A similar decrease was seen in the percent representation in CD4 and CD8+ T-cells (lower panels). Conversely, an increase in the percent representation of TCF1-cells was observed in severe patients. Similarly, there was a decrease in the MFI values for T-cells amongst the TCF1 positive population from 472 to 168 (FIG. 6B, upper panel) and in CD4+ and CD8+ cells (lower panels). viSNE analysis confirmed the longitudinal changes in expression of TCF1 from a severity of WOS 4 to 6 (FIG. 6C). The pattern showed 5 clusters in the CD4+ cells with the majority of TCF1 staining in clusters 1 and 3 in samples from patients with severity 4. There was a dramatic loss of TCF1 expression and cells in the T-cells from severity 6. Instead, a new island (cluster 5) arose with fewer numbers of cells with TCF1 expression (also see lower histogram). Similarly, viSNE analysis of CD8 cells showed a pattern with 4 clusters (upper right panel) with reduced expression on islands 1 and 2, accompanied by an increased number of cells in islands 3 and 4 with markedly reduced TCF1 expression (also see lower histogram).

SPADE provided further detail and power in identifying different nodes of CD4+ and CD8+ T-cells expressing TCF1 (FIG. 6D). From this, over 70 subsets of cells were seen where TCF1 expression was seen with severity 4 with tree grouping iii (left panel). The transition to severity 6 different levels of TCF1 expression were seen in the various trees of CD4 cells with different nodes (see arrows i-v). The pattern with WOS severity 6 showed a shift in the presence of cells to tree grouping iv with markedly reduced TCF1 expression (right panel). A similar change was seen by SPADE of CD8+ cells expressing TCF1 (FIG. 6E). Nodes with intermediate-high TCF1 expression during the period of severity 4 (v and iii) were lost when the patient progressed to severity 6 accompanied by an increase in nodes with low expression (tree grouping (vi and ii) and the loss of cells which failed to express TCF1 (vi and i). Overall, these representations showed that a diversity of CD4 and CD8 cells expressing intermediate-high TCF1 lost this expression during the transition from severity 4 to severity 6.

Lastly, Spearman analysis of the foldincrease in severity of several patients in a longitudinal study showed a marked decrease in the delta CD8 with a striking r value of-0.71 and a p value of 0.08 (left panel) and in the fold decrease in CD8+ TCF+ T-cells with a striking r value of −0.8982 and a p value of 0.0095 (FIG. 6F).

### Catastrophic Loss of Cell Division in TCF1+ T-Cells in Severe Disease

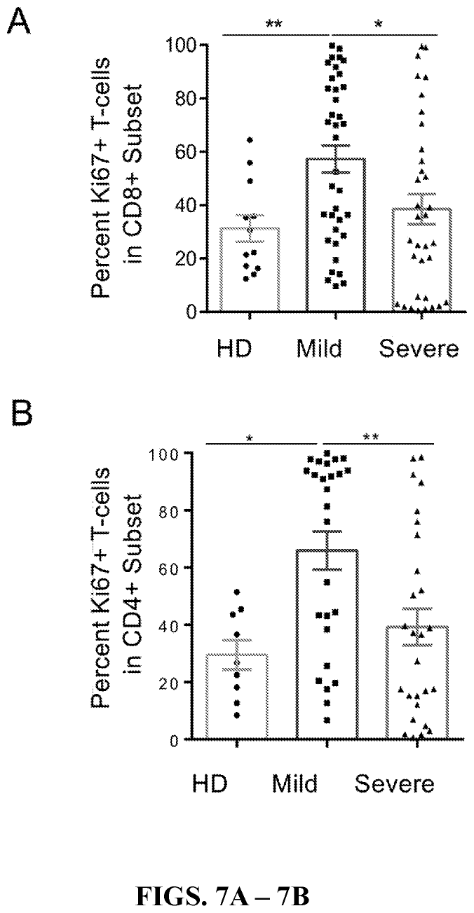

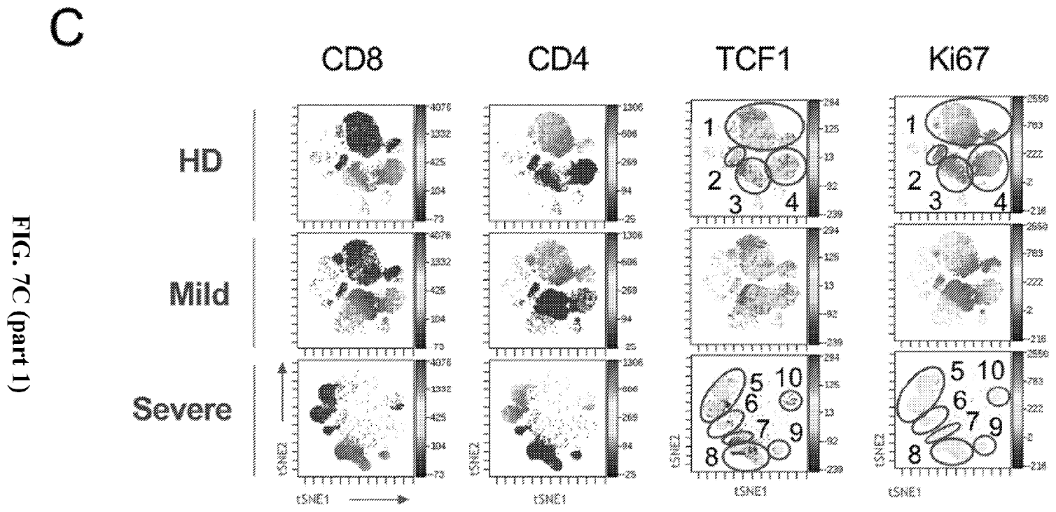

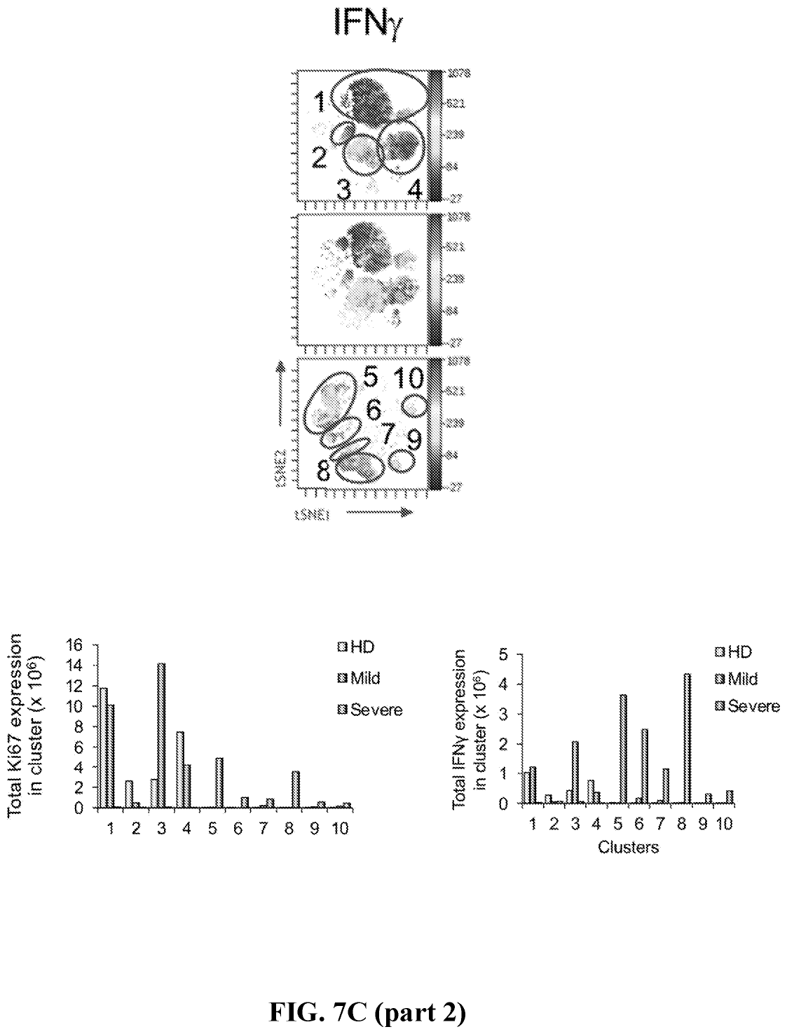

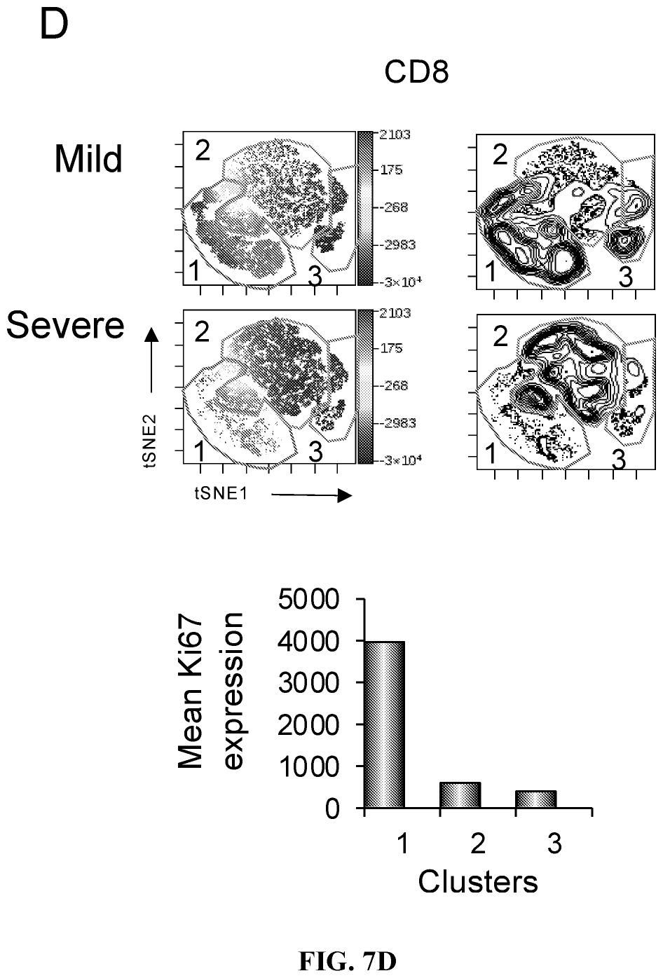

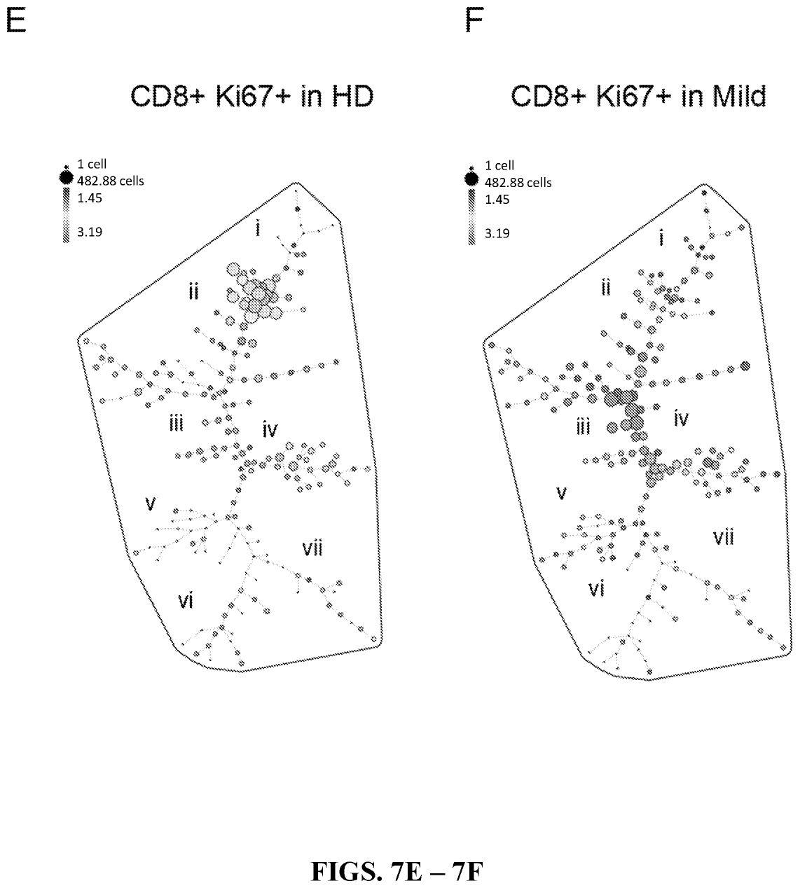

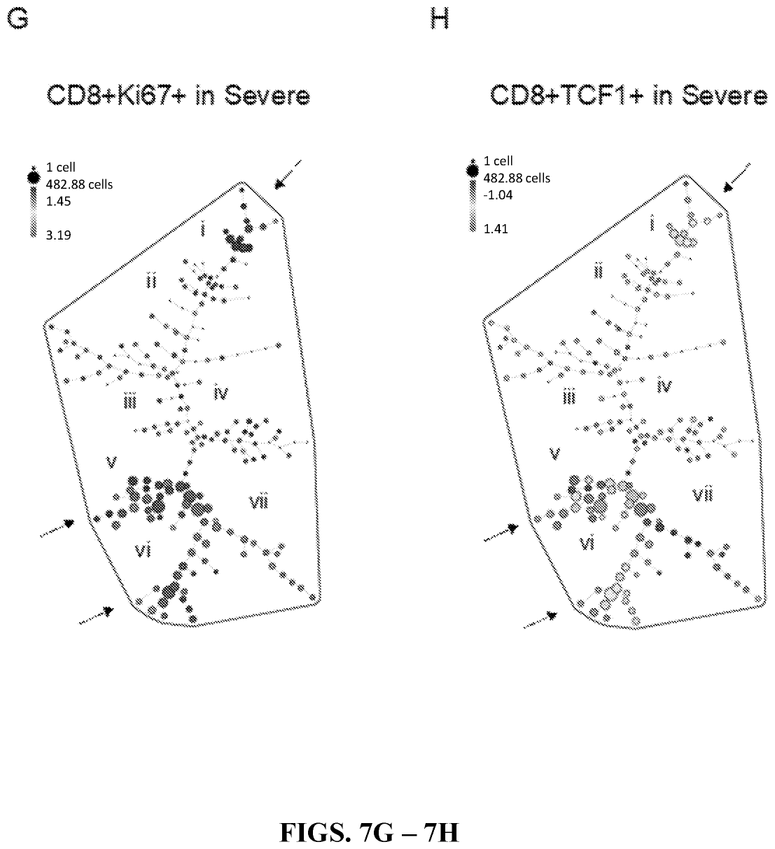

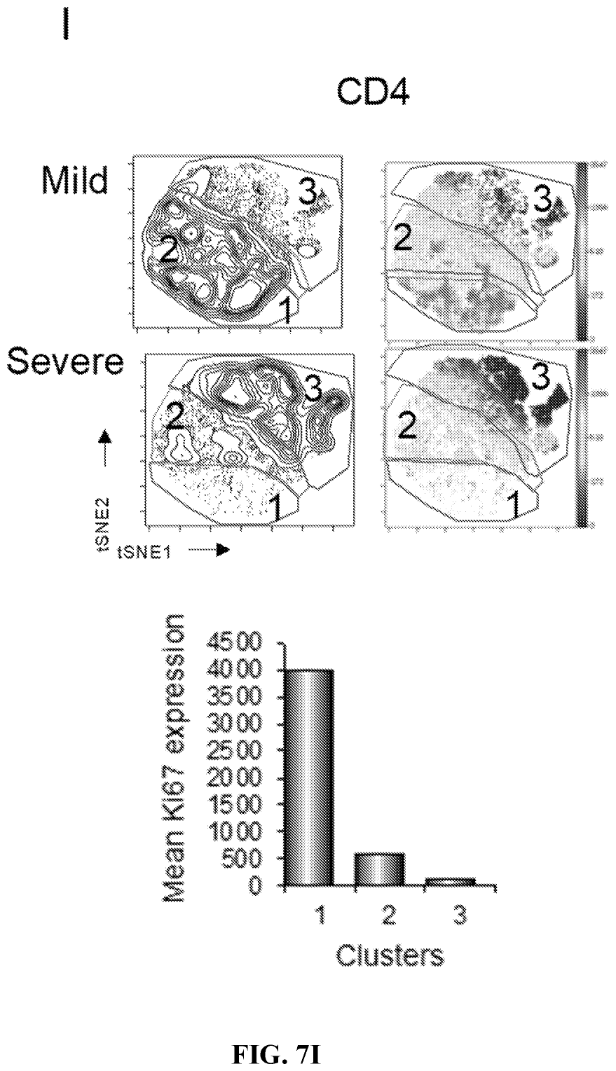

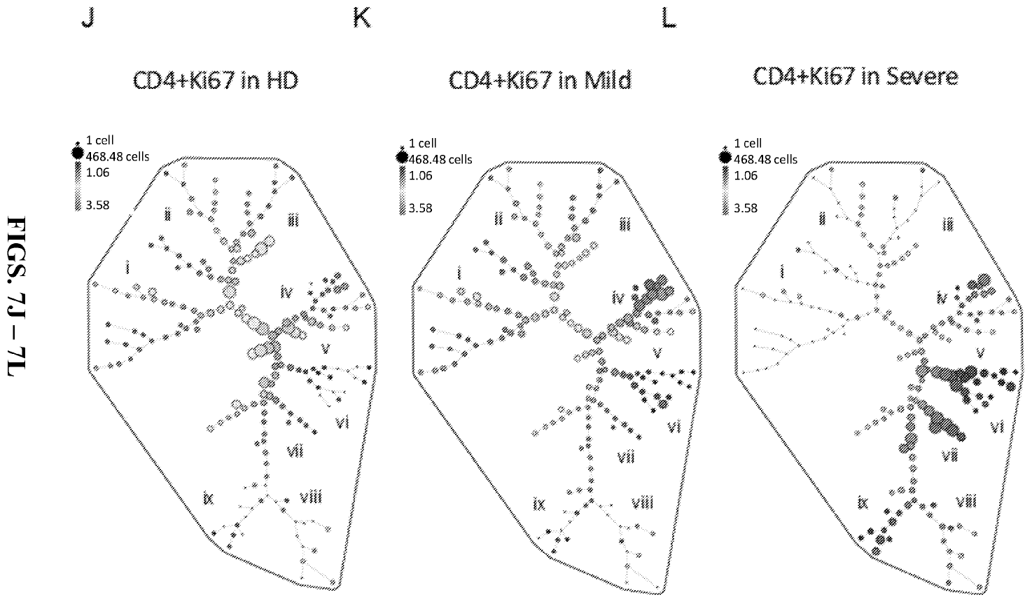

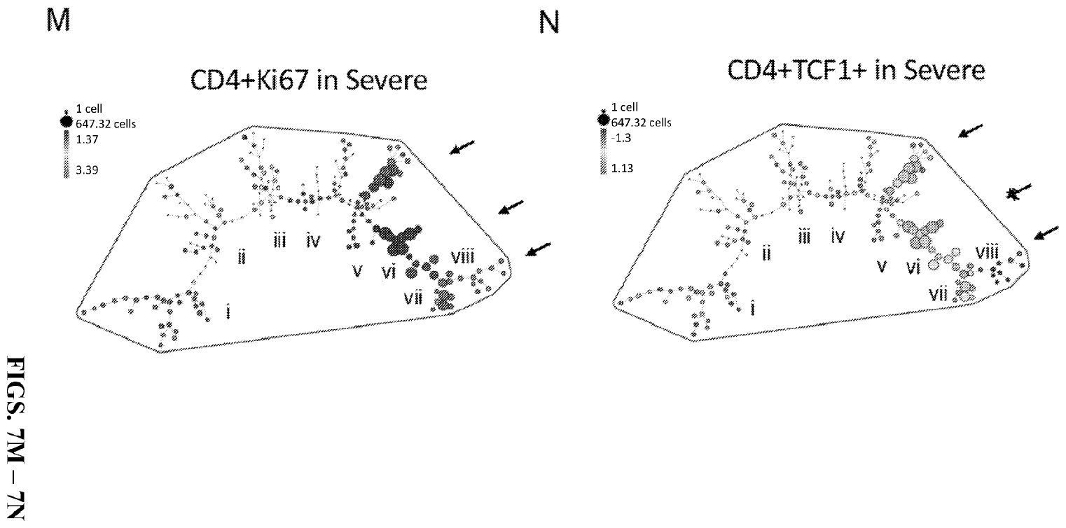

One possibility was that the loss of TCF1+ T-cells might be accompanied by a loss in the ability to proliferate and clonally expand (FIG. 7). To explore this, Applicant next monitored the expression of Ki67, a well-established marker for cell cycling and proliferation (42). The protein is highly expressed in cycling cells, but then is markedly downregulated in resting G0 cells. Applicant first observed a significant reduction in the presence of CD8+Ki67+ (FIG. 7A) and CD4+Ki67+ T-cells (FIG. 7B) in samples from patients with severe vs. mild disease. Applicant observed an increase in the presence of Ki67+ CD4 and CD8 T-cells in mild patients relative to healthy donors as would be expected in a response to infection. This finding is consistent with previous reports showing increases in the presence of Ki67+ subsets in COVID-19 patients (6, 13, 56, 57). However, in Applicant's hands, this increase was limited to patients with mild disease. However, by contrast, severe disease was accompanied by a marked loss of Ki67 expression indicative of loss of cell proliferation and cycling.

viSNE analysis of CD3+ T-cells confirmed the loss of Ki67 among TCR−β+ T-cells (FIG. 7C, upper panel). This included CD4+ cluster 1 which shifted in position to become cluster 5 in severe patients, CD8+ clusters 3 and 4 which became islands 7-9 and CD4−CD8− cluster 2 which became cluster 6. In each case, the newly formed clusters in samples from severe patients showed a dramatic loss of Ki67 staining (also see lower histogram). The pattern change was similar for TCF1 staining except for islands 7 and 10 which retained moderate levels of expression (as seen by red staining). For comparison, Applicant also examined anti-IFN-γ staining which showed an increase in expression in clusters 5, 6, 7 and 8 from patients with severe disease (right upper panel and lower right histogram). This indicates that although severe disease is associated with a higher levels of effector cytokine production, the process occurs concurrently with a loss of TCF1 and Ki67 expression indicative of cell division.

In order to obtain a better resolution, Applicant next conducted viSNE and SPADE analysis on gated CD8+ cells (FIGS. 7D-M). A representative sample of CD8+ cells showed a high level of Ki67 staining in island 1 which was lost in the sample from severe patients with the appearance of a greater number of cells in cluster 2. The expression of Ki67 on the cells in cluster 2 showed negligible staining (also see lower histogram). SPADE analysis further showed a striking impact of severe disease on the expression of cell cycling in CD8+ cells as defined by Ki67 expression (FIG. 7F-H). This panel represents the concatenated samples from 9 patient samples for each group. From this, Applicant was able to subdivide the population into many subsets (>100) based on the expression of markers. Among these, Applicant further separated cells in 7 tree groupings (i-vii). The nodes in samples from HDs showed low levels of Ki67 expression (i.e., mostly light pink color) (FIG. 7E), which increased in cells from patients with mild disease. This increase in TCF1 expression was seen mostly in tree clusters iii and iv (FIG. 7F). However, in striking contrast, the expression of Ki67 was almost completely lost in the array of nodes within the CD8+ subset (FIG. 7G). Samples from patients with severe disease also show the contraction of nodes iii and iv accompanied by an increase in tree groupings i, v-vii. Each of these node clusters showed a catastrophic loss of Ki67 expression indicative of a loss of proliferation and cell division (see arrows). A comparison with TCF1 staining showed that the loss of Ki67 occurred in many of the cells and nodes that stained positively for the transcription factor (i.e., tree clusters i, iii, iv, v, vi and viii) (FIG. 7H). In certain cases, Applicant observed a loss of Ki67 staining in cells which had also lost TCF1 expression (i.e, vii). These data confirmed that severe disease is linked to a remarkable loss of proliferative capacity in the CD8 peripheral T-cells.

Applicant next examined expression in the CD4+ subset (FIG. 7I-N). viSNE showed high levels of Ki67 expression in cluster 1 in cells from patients with mild disease that was strikingly lost and replaced by an increase of cells in cluster 3 with negligible levels of Ki67 expression in severe patients (also see lower histogram). SPADE analysis confirmed the catastrophic loss of Ki67 expression in most subsets of CD4 (FIG. 7J-N). In the pattern from HDs, Applicant observed the expression of Ki67 primarily in tree groupings i-iii. In the case of mild patients, Applicant observed a slight increase in staining intensity in these groupings combined with the loss of expression in other groupings (i.e., iv and v). However, in the case of severe disease, as Applicant observed in the case of CD8 cells, there was a contraction in the size of nodes in groupings i-iii combined with an increase in the size and the loss of Ki67 staining in groupings iv, v, vi, vii, viii and ix. A similar loss of expression was also seen when SPADE was used to assess other patient samples (FIG. 7M) which clearly overlapped with detectable TCF1 staining (FIG. 7N vs. 7M). Overall, these data confirmed that severe disease is linked to a remarkable and potentially catastrophic loss of the proliferative capacity of TCF1+ CD8 peripheral T-cells.

Reduced Expression of Pro-Survival Bcl-2 in T-Cells from Patients with Severe Disease is Accompanied by the Appearance of Caspase 3

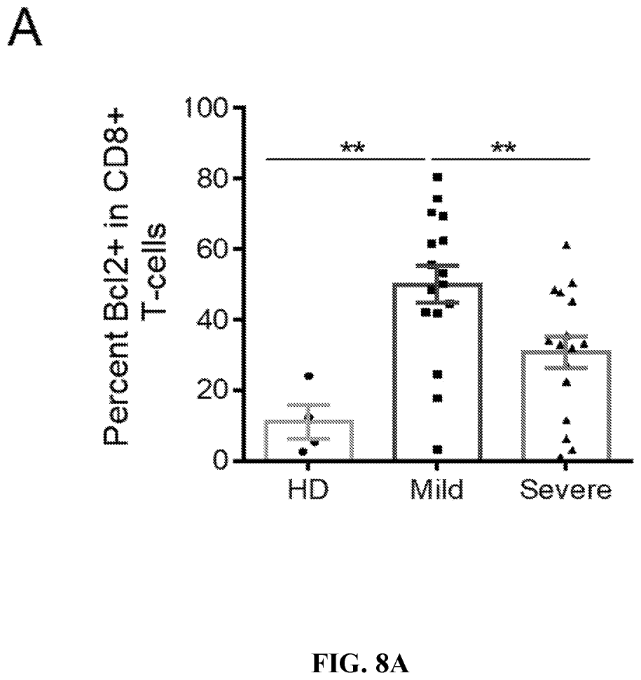

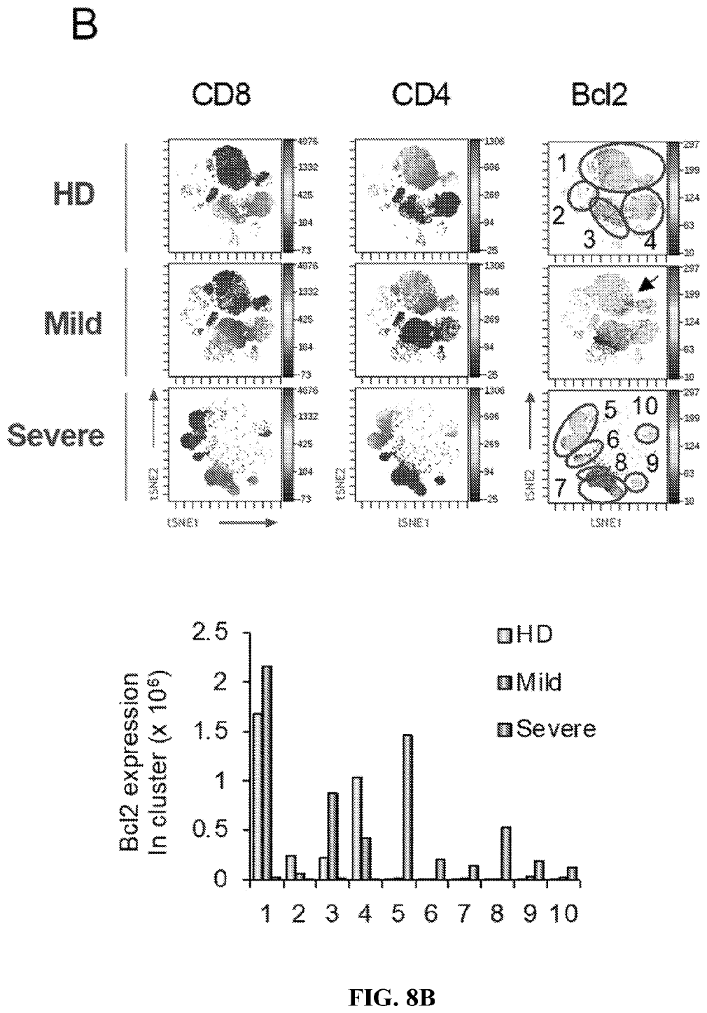

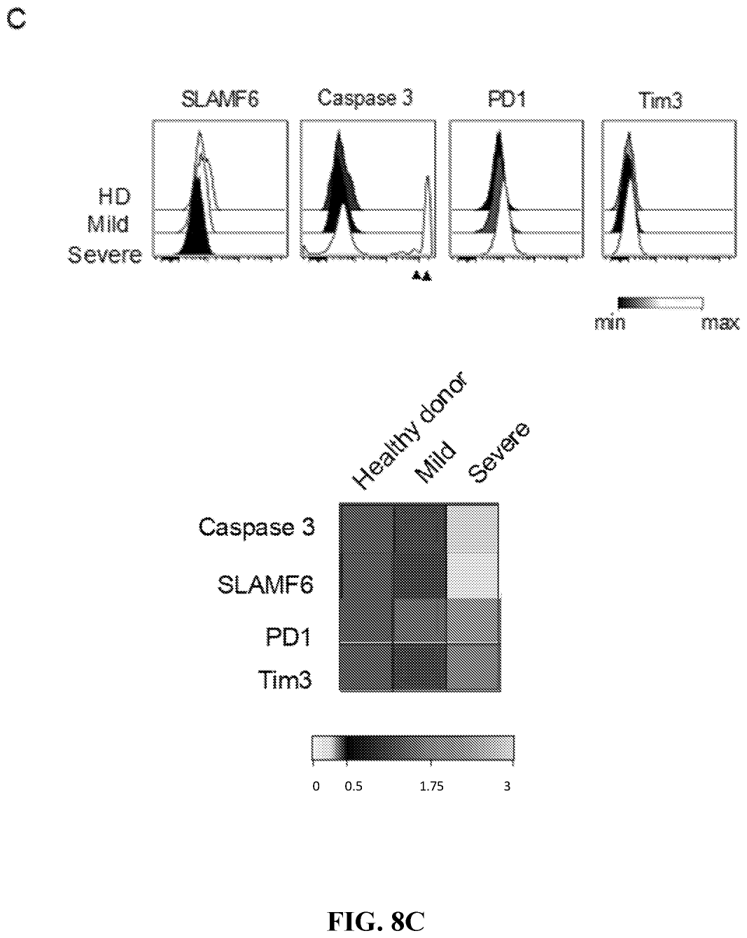

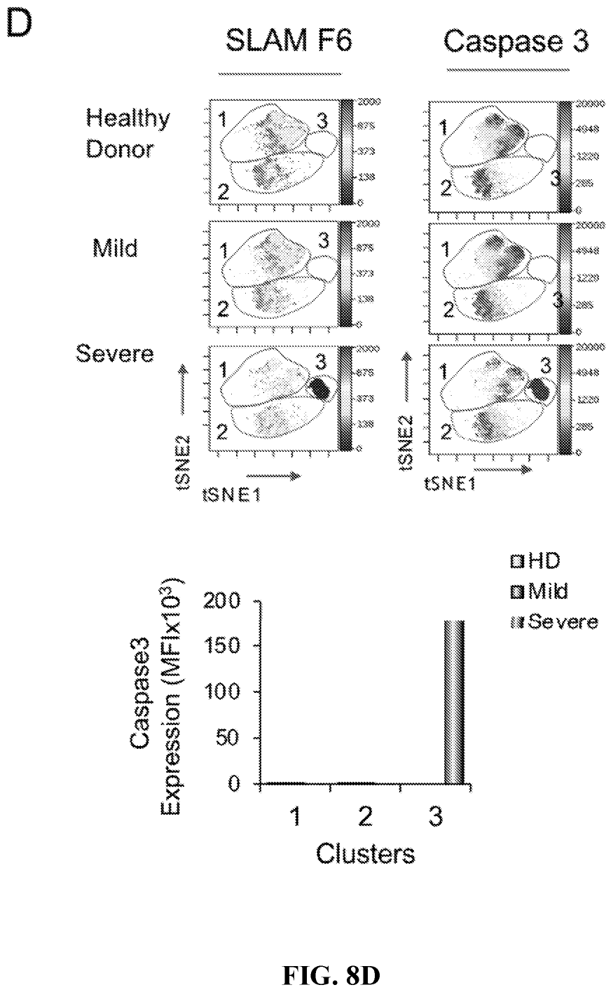

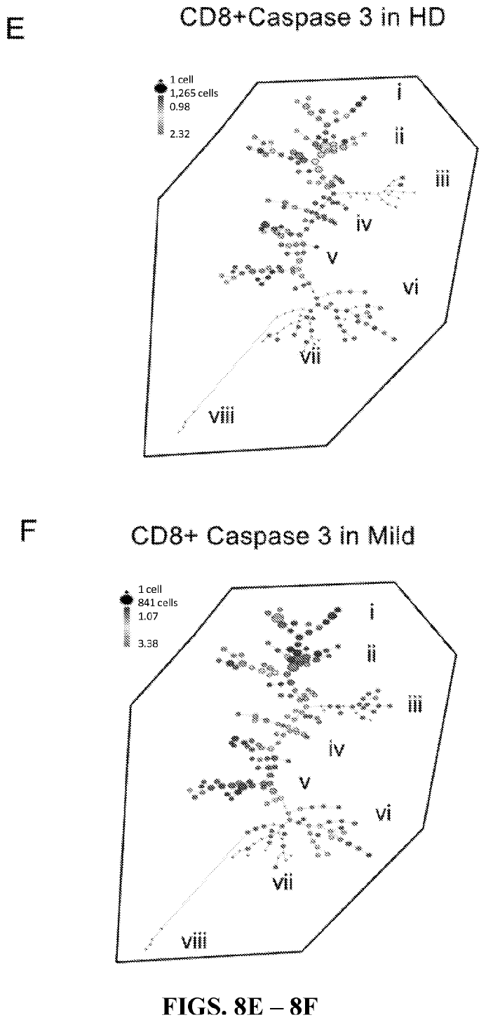

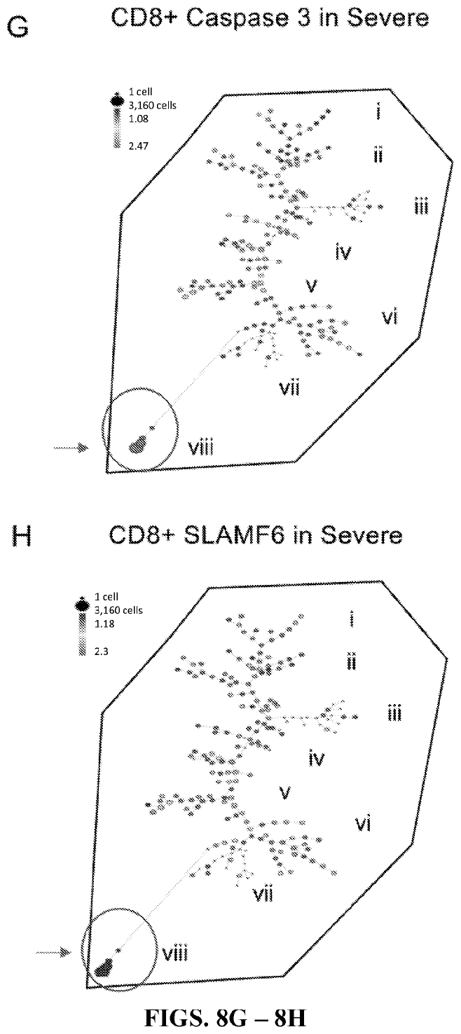

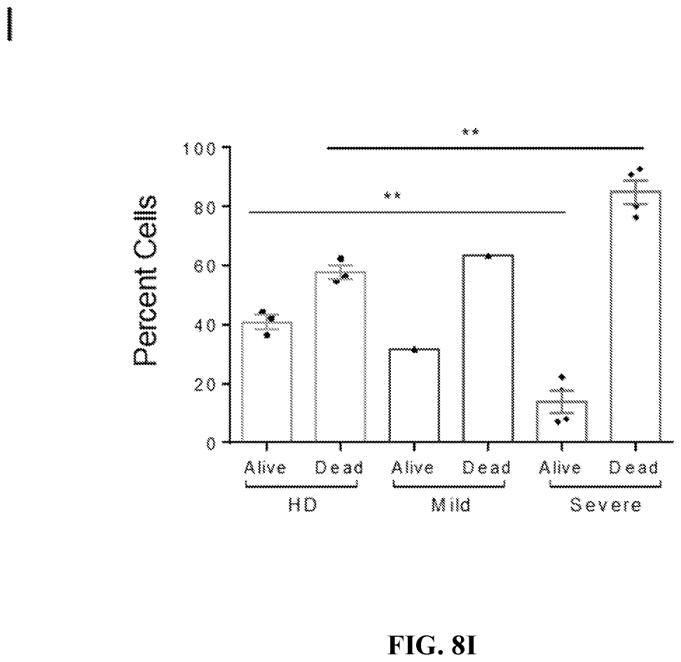

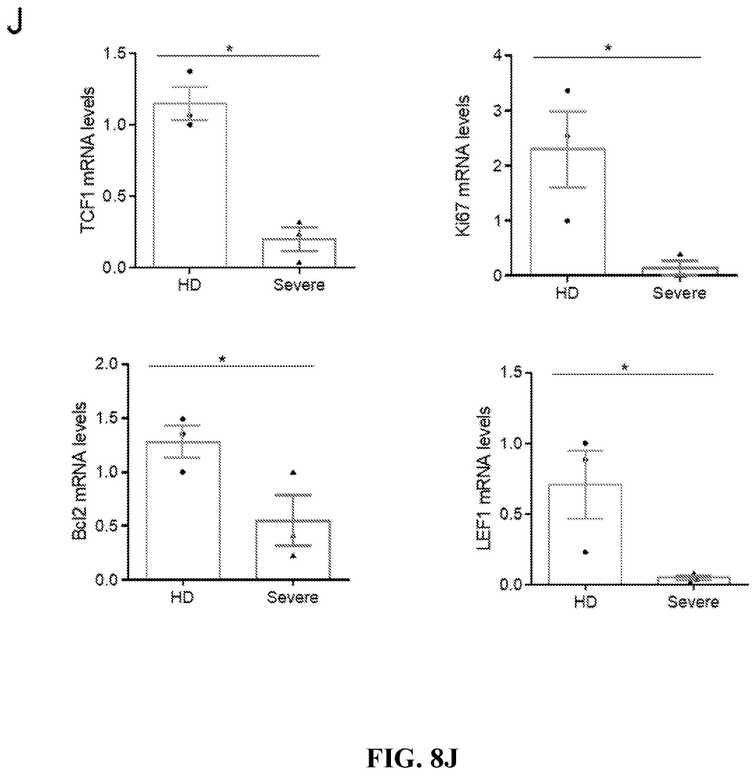

Given the striking loss of cell division, Applicant next asked whether the loss of TCF1+ and Ki67 was associated with an in increase in cell death. B-cell lymphoma-2 (Bcl2) is a pro-survival factor needed for the survival of stem cells and T lymphocytes (61). With a focus on the CD8+ T-cells, Applicant found that the expression of BcL2 increased in patients with mild disease relative to HDs (FIG. 8A). This is consistent with the need for increased cell survival during the response to SARS CoV2 antigens. However, by contrast, there was a significant loss of BcL2 expression in CD8 cells from patients with severe disease. viSNE analysis confirmed the moderate anti-Bcl2 staining of islands of CD4 and CD8 patterns from HDs followed by an increase in expression (i.e., cluster 1) from patients with mild disease (FIG. 8B). However, this increase was lost in the clusters from patients with severe disease (also see right histogram). These data showed that the anti-apoptotic factor Bcl2 was markedly reduced in CD8+ cells.

Given this loss of pro-survival Bcl-2, Applicant next examined whether there was an increase in the expression of the pro-apoptotic factor caspase 3. Firstly, Applicant examined expression using conventional flow cytometry with antibodies to SLAMF6, a surrogate for TCF1 expression (65). Consistent with TCF1 expression, Applicant observed a decrease in expression in CD8+ T-cells from patients with severe disease (FIG. 8C). At the same time, Applicant observed a slight to moderate increase in PD1 and a slight increase in TIM3. However, intriguingly, caspase 3 staining showed the appearance of two new peaks with high levels of caspase 3 expression only in the cells from patients with severe disease (see arrows). This was only observed in samples from severe patients. The Heat map also showed the inverse relationship between the expression of TCF1 and caspase 3 (lower panel).

Applicant also gated on the SLAMF6 population, a surrogate marker for TCF1 expression, which confirmed the loss of expression in CD8+ T-cells from severe patients (FIG. 8D, upper left panel). Brightly labelled clusters 1 and 2 in HDs and mild patients showed reduced staining in severe patients that was accompanied by the appearance of a new cluster 3 with no SLAM6 staining. Intriguingly, this shift was accompanied by the appearance of a new cluster which was brightly stained for caspase 3, an indicator of cell death (upper middle panel). Histograms show that cluster 3 had high levels of caspase 3 (lower panel).

SPADE analysis revealed that, in samples from patients with mild disease, caspase 3 shows low level expression in CD8+ cells (see tree groupings i, ii, iv and v) (FIGS. 8E,F). The larger negative nodes in patients with mild disease are consistent with increased cell survival. By contrast, the pattern for patients with severe disease underwent a noticeable contraction of numbers of cells accompanied by the appearance of a large node at the bottom of the pattern (FIGS. 8G, see arrows). The same node failed to express TCF1 (FIGS. 8H, see arrows). These data are consistent with a contraction and loss of TCF1 expression accompanied by the cell death of a sizable portion of peripheral CD8+ T-cells in patients with severe disease.

Ex Vivo Cultures Showed Increased Cell Death and the Sustained Loss of TCF1, Ki67, Bcl2 and LEF1 Expression in T-Cells from Patients with Severe Disease.

To explore this further, Applicant next attempted to culture peripheral T-cells from patients and to measure their loss from culture (FIG. 8I). 200,000 cells/ml were cultured for 5 days in RPMI 1640 media with 10 percent FCS and cell numbers were measured. No additional signal was added. After 6 days, Applicant observed a decrease in numbers of T-cells from mild vs. severe (i.e., 20,000 cells) (left panel). The capacity of CD4 and CD8 cells to expand ex vivo was also impaired especially in the case of CD8+ cells. Gating on the live and dead cells also showed that peripheral T-cells had a much greater % of dead cells (i.e., 80%) relative to HD and mild samples (FIG. 8I).

At the same time, Applicant monitored the expression of TCF1, Ki67, Bcl2 and LEF1 by PCR (FIG. 8J). Cells were cultured for 6 days before the measurement by qPCR. Although there was cell death, there were also sufficient cells to measure mRNA expression by PCR. In each case, Applicant saw a striking loss of TCF1, Ki67 and LEF1 mRNA expression in the cells from patients with severe disease. A reduction in the expression of Bcl2 was also observed. Collectively, these data indicated there was a profound loss in the ability of TCF1+ (and TCF1-) T-cells to proliferate in patients with severe disease. A trend for impaired proliferation was also seen in patients with mild disease; however, the trend became statistically and markedly profound in both the CD4 and CD8 cells of severe patients.

### The Loss of TCF1+ is Sustained and Induced by the Proinflammatory Cytokine IL-12.

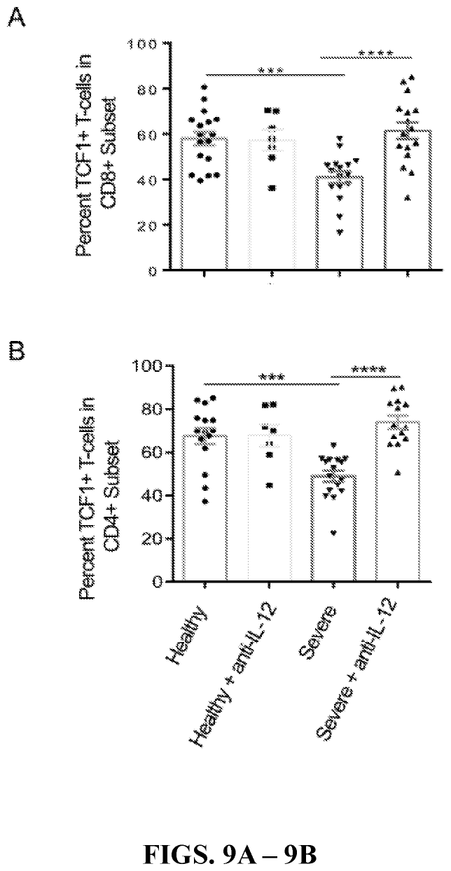

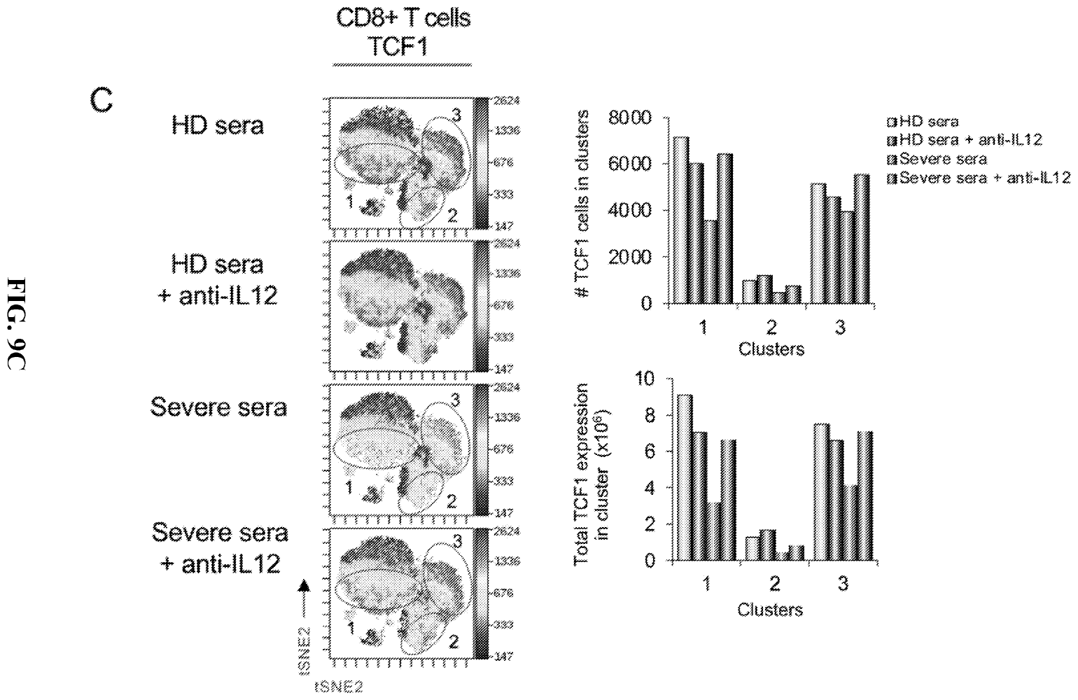

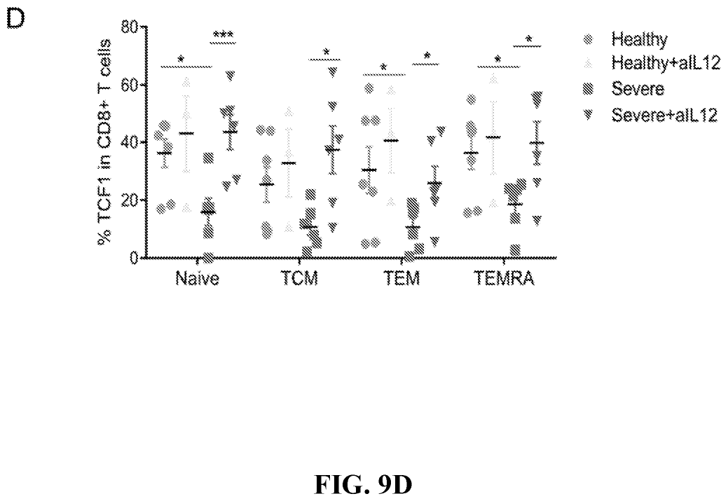

Lastly, COVID-19 severe disease is associated with a severe cytokine storm involving pro-inflammatory interleukins such as IL-1α, IL-1β, IL-6, IL-8, IL-12, IL-17, and TNF-α (tumor necrosis factor-α) (66). Intriguingly to us, IL-12 has been reported to down-regulate TCF-1 at the transcriptional level (67). To assess whether IL-12 in the plasma of severe patients was responsible for the loss of TCF-1+ expression in T-cells, Applicant incubated peripheral T-cells from healthy donors to aliquots of sera from healthy donors or patients with severe disease for 6 days followed by an assessment of TCF1 expression (FIG. 9). Remarkably, the sera from severe patients reduced TCF1 expression in CD8 and CD4+ T-cells and further, the co-incubation of a blocking antibody to IL-12 reversed this decrease (FIG. 9A, B). viSNE analysis showed a marked reduction in several clusters of TCF1+ cells. Importantly, the addition of a blocking anti-IL-12 antibody reversed the loss of TCF1 induced by the sera from severe patients (FIG. 9C). Finally, the inhibition of TCF1 transcription was observed in naïve, TCM, TEM and TEMRA cells and was reversed by anti-IL 12 in all subsets (FIG. 9D). This data strongly suggested that the production of IL-12 in the inflammatory cascade induced in severe disease was responsible for the inhibition of TCF1 expression and ultimately the loss of T-cells from the circulation of patients with severe disease.

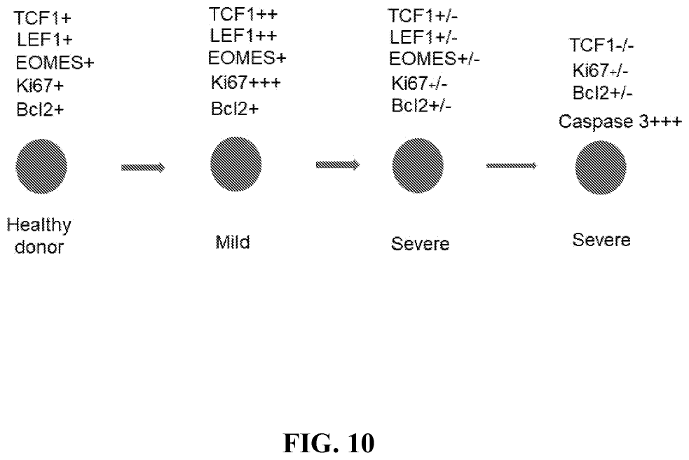

Overall, as shown in FIG. 10, Applicant has shown that the transition from mild to severe COVID-19 is characterized by the preferential reduction of TCF1+ and Ki67 expression in CD4+ and CD8+ T-cells in patients with severe disease. This in turn involves the preferential loss of TCF+ T-cells relative to TCF1-T-cells in the peripheral blood of patients with severe disease. A comparison of healthy donors with patients with mild disease showed an increase in Ki67 expression on TCF1+ and TCF1-T-cells indicative of an increase in proliferation and an enhanced immune response against SARS-CoV2 infection. By contrast, a marked loss of Ki67 expression was seen in TCF1+ and TCF1-CD4+ and CD8+ T-cells from patients with severe disease. A loss of Ki67 in TCF1+ T-cells indicates that the progenitor T-cells had a markedly reduced capacity to undergo cell division. A loss of other markers such as the progenitor transcription factor EOMES in T-cells was observed, as well as the progenitor transcription factor LEF1 and the progenitor marker Notch 1 in CD8+ T-cells. The loss of proliferation of the progenitor stem-like population is needed to replenish the immune system. This transition from mild to severe disease was further accompanied by a reduction in the expression of pro-survival BcL2 resulting in the final stage of an increase in caspase 3 expression (cell death) especially in the TCF1-cells. The loss of these factors was seen by flow cytometry and quantitative PCR. A marked reduction in the transcription of TCF1, Ki67, LEF1 and Bcl2 in the overall T-cell population from patients with severe disease was also seen. Without being bound by theory, this is why the immune system collapses with a large loss of T-cells and mortality.

Overall, Applicant's data supports a model that accounts for the dramatic loss of T-cells in severe disease where TCF1+ T-cells are needed to replenish T-cells in the peripheral immune compartment. The preferential depletion of TCF1+ T-cells was evident from a comparison of the relative presence of TCF1+ versus TCF1-T-cells in the peripheral T-cell population in blood from patients with severe disease. This difference has not been observed in previous studies (5, 13, 56, 57, 68, 69). Further, a comparison of patients with mild versus severe disease showed a statistical difference in the percent and the MFI of expression of TCF1+ in CD8 T-cells in patients with severe versus mild disease. This was observed both cross-sectionally and longitudinally and involved the loss of TCF1+ on all subsets of CD8+ T-cells and most CD4+ cells. viSNE and cross-sectional analysis further showed a loss of the cell cycle indicator Ki67 and the pro-survival mediator Bcl2 in the TCF1 depleted cells, accompanied by the appearance of the death marker caspase 3 in TCF1^{lo}-negative cells. These data showed that the CD8+ T-cells with reduced TCF1 were no-longer dividing and instead were progressing to cell death. Interestingly, a loss of Ki67 was seen in both TCF1+ and TCF-cells suggesting a more global inhibition of cell cycling in CD8 cells; however, the loss in TCF1+ cells should prevent the renewal and refurbishment of new cells in the depleted T-cell compartment. Further, cell death as identified by the appearance of caspase 3 was preferentially seen in the TCF1-depleted subset of cells.

Applicant first noted an unexpected preferential depletion of CD8+ and CD4+ TCF1+ T-cells from the peripheral blood of patients with severe disease. This loss was correlated statistically with increasing WOS severity scores. Further, when patients were grouped into mild (WOS score of 1-4) versus severe disease (WOS score of 5-8), Applicant noted a clear statistical reduction in the presence of peripheral CD8+ and CD4+ TCF1+ T-cells. The decline was seen in the MFI of TCF1 expression and in the percent representation of TCF1+ CD8 T-cells in peripheral blood. Further, the loss was seen in both cross-sectional and longitudinal analysis, where for example, a shift in WOS scoring of 4 to 6 was accompanied by a loss of TCF1 expression. The loss of TCF1 expression was also seen in a range of different subsets that included human central memory (TCM, CCR7+CD45RA−), effector memory (TEM, CCR7−CD45RA−), and effector memory cells re-expressing CD45RA subset (TEMRA, CCR7−CD45RA+). In this sense, TCF1 plays a critical role in maintaining protective immunity (33). Further, under normal circumstances, the central memory cells retain a proliferative capacity to regenerate and produce differentiated cells and as such, are needed to sustain the immune response to chronic infection (34). Overall, these observations indicated that TCF1 expression and TCF1+ expressing T-cells are preferentially downregulated with an increasing severity of COVID-19 disease.

In a normal immune response, self-renewing TCF1+ cells give rise to TCF 1^{hi} expressing self-renewing cells and TCF1^{lo} effector populations (70). This pattern was observed in the response of mild patients to infection with SARs CoV2. However, in the case of severe COVID-19 disease, the process is compromised by a striking loss in the proliferative capacity in TCF1+ CD4 and CD8+ T-cells as seen by the loss of Ki67 expression. This was evident in a comparison of the presence of CD4+ TCF1+ and CD8+TCF1+ subsets in a screen of multiple patients with severe vs mild disease (>60), and was visualized by conventional flow, viSNE and SPADE analysis. Ki67 is a well-established marker for cell cycling and proliferation since it is down-regulated in resting G0 cells (42). As mentioned, its expression is widely used as a measure of the proliferation index that determines treatment decisions (43). The loss of Ki67 expression in severe disease was significantly different when comparing groupings of patients with severe versus mild disease or with healthy donors, a pattern also seen for TCF1 expression. Interestingly, the T-cells from patients with mild disease showed an increase in Ki67 expression in mild diseased patients compared to healthy donors as would be expected for productive proliferative responses against a SARS-CoV2 infection. It is also consistent with a study that focused on CD4+ subsets with Ki67 expression such as the CD8+ subset CD38+HLA−DR+Ki67+ (5).

However, by contrast, the pattern changed markedly in patients with severe disease where the proliferative response was dramatically terminated with the loss of Ki67 expression in CD4 and CD8 subsets as viewed graphically in viSNE and SPADE analysis. In viSNE, there was the replacement of cells in a cluster with high Ki67 expression accompanied by the appearance of a new cluster with greatly reduced expression. SPADE analysis of T-cells in patients with severe disease also showed a redistribution of cell clusters in various tree groupings and nodes with widespread reduction in Ki67 expression. The was observed in both CD4 and CD8 cells where in the case of CD8+ T-cells, there was a loss of expression in 98 of 105 nodes in the SPADE pattern. Further, the loss of Ki67 overlapped with the nodes that showed reduced TCF1 expression.

In this context, the contraction and loss of TCF1-Ki67 expression was also accompanied by a reduction in the expression of the pro-survival mediator BcL2, a founding member of a family of mediators that control cell survival (44). Although the reduced expression is less pronounced than seen for TCF1 and Ki67, it was still statistically significant and pronounced when assessed by qPCR and FACs. The reduced expression in severe disease grouping (WOS score of 5-8) was also statistically significant relative to the mild (WOS score of 1-4) grouping. Its expression also generally increased in mild patients relative to healthy donors, consistent with a proliferative response to infection; however, this increase was then reduced in cells from severe patients. Further, Applicant observed that appearance of a new population of CD8+ cells lacking TCF1 expression but having markedly increased caspase 3 expression indicative of cell death. As seen in viSNE and SPADE patterns, this new cluster of caspase-3 high expressing cells in severe disease was located in the same population that that had lost TCF1 and Ki67. Further, consistent with a loss of viability, the ex vivo culturing of peripheral T-cells from severe patients showed greater cell death after days in culture than peripheral T-cells taken from healthy donors and patients with mild disease. The same cells showed a dramatic loss in the mRNA expression of TCF1, BcL2 and Ki67 expression as measured by qPCR. The same population was also seen to express caspase 3, a marker for cell death. To Applicant's knowledge, the detection of a specific caspase 3+ population in the peripheral blood from patients with severe disease has not been previously reported. An increase in caspase 4 associated with macrophage function in mouse models of infection has been reported in a non-peer-reviewed paper (71). Applicant's observations complement the definition of other key changes in the immune system (5, 13, 56, 57, 68, 69)

The ex vivo culturing of T-cells from severe patients failed to show any sign of TCF1 re-expression as defined by qPCR, while importantly, the addition of sera from patients with severe disease caused the loss of TCF1 expression in cells, an effect prevented by the addition of anti-IL-12. Applicant's findings introduce a model to explain the dramatic loss of peripheral T-cells in severe COVID-19 disease where the preferential loss of self-renewing progenitor TCF1+ T-cells by inflammatory cytokines such as IL-12 leads to their death and the collapse of the immune system in severe disease. It could explain the well-documented loss of T-cells and reduced antibody responses in patients with severe COVID-19 disease (5, 6) and the loss of CTLs in respiratory tract in patients with severe disease (17). Although some peripheral lymphopenia may be due to the recruitment of lymphocytes to the inflamed respiratory vascular endothelium, lung autopsy studies suggest that lymphocytic infiltration is not excessive (24, 25). Applicant's model would argue that the loss of T-cells in the peripheral compartment is due to the loss of progenitors needed for an immune response. TCF1 axis with Bcl6 counteracts type I interferon to repress T cell exhaustion and maintain T cell stemness, which is critical for persistent antiviral CD8 T cell responses in chronic infection (38). It could also account for the loss of antibodies in severe disease since the loss of TCF1 promotes T cell differentiation to Th2 cells in the periphery through transcriptional activation of GATA3 (72). Th2 T-cells provide help needed for antibody production and facilitate tissue repair (73).

Applicant's findings also indicate that the testing of the expression of these key mediators by an assessment of protein expression or transcription should identify patients with different stages of disease severity. In particular, it should allow for the identification of patients with severe life-threatening disease prior to the full manifestation of clinical symptoms. Their expression should also allow for the prediction of whether a patient with mild disease may progress to severe disease or will remain stable with mild disease. This would involve assessing protein expression by various means such as flow cytometry or transcription via other means such as qPCR for TCF1 expression combination with Ki67 and Bcl2 expression. The progression to severe disease would be expected to involve the loss of TCF1 and Ki67 expression combined with reduced Bcl2 expression and the appearance of T-cells with the increased expression of caspase 3. It might also involve the loss of Ki67 expression in both TCF1+ and − T-cells or the potential loss of TCF1 without the loss of Ki67 and Bcl2. The loss of TCF1 expression could precede the loss of Ki67 expression or vice versa. Further, the loss of other progenitor markers such as LEF1 and Notch1 could complement and increase in the power of detection of the loss of progenitor T-cells in combination or distinct from the loss of TCF1 or Ki67 expression. The particular combined reduction in TCF1, Ki67, Bcl2, LEF1 and Notch 1 expression as an indicative or predictive marker may also vary from patient to patient. Eomes expression was also reduced in the case of patients with severe disease.

Lastly, in severe disease, lymphopenia had been correlated with high levels of inflammatory cytokines (11, 15, 16, 19). In this context, IL-12 can downregulate TCF1 by inhibiting DNA methyltransferases (67). This observation fits well with Applicant's observation that anti-IL12 was capable of preventing the downregulation of TCF1 in response to patients with severe disease. Applicant's study suggests that the use of anti-IL-12 blocking antibodies such as STELARA® (ustekinumab), presently used to treat psoriasis and Crohn's disease (CD)) (49) might also prevent the loss of T-cells in severe disease. Further, in this context, the anti-inflammatory glucocorticoid, dexamethasone, which has proven successful in treating COVID-19 patients (50), also inhibits IL-12 production (51).

### Methods:

### Patients and Clinical Data Collection

In line with the McGill University Health Centre Research Institute (MUHC-RI) Ethics Board approved human study (#2021-6081), informed consent was obtained from patients admitted to the McGill University Health Centre (MUHC) with PCR confirmed SARS-CoV-2 infection between April 2020 and March 2021 (n=76). Peripheral blood samples (K2EDTA-preserved whole blood and/or sera were obtained from this patient cohort and from healthy adult donors (HDs) with no prior diagnosis of or recent symptoms consistent with COVID-19 disease. Clinical data was recorded into standardized case report forms. Median time from patient admission to collection of initial PBMC samples was 3 days (IQR 1-8 days). Serial samples from a subset of patients (n=18) were collected at time points out to 36 days from admission. Clinical outcomes and severity of COVID-19 manifestations were assessed using the World Health Organisation (WHO)'s COVID ordinal scale. Scores of <=4 were considered to be mild manifestations of disease and >=5 were considered to be severe. Clinical laboratory data was abstracted from the time points closest to that of research blood collection as well from time points associated with extreme values. Additional peripheral blood samples from uninfected healthy adults were obtained from the Hema-Quebec blood bank as approved by the CR-HMR Ethical Approval (Le Comité de protection des animaux du CIUSSS de l'Est-de-l'Île-de-Montréal (CPA-CEMTL), F06 CPA-21061 du project 2017-1346, 2017-JA-001).

### Sample Processing

The peripheral blood mononuclear cells (PBMCs) were isolated at the MUHC-RI using the density gradient centrifugation method. Peripheral blood was collected into K2 EDTA (BD) tubes and layered above an appropriate amount of density gradient in 15 ml tubes (Lymphoprep by Stemcell Technologies). These were spun for 30 minutes at 400 g at room temperature (RT). Plasma was collected and banked. The PBMC layer was collected and washed twice with PBS. Isolated PBMCs were stained for viability and counted, then fixed in 2% paraformaldehyde at room temperature for 20 minutes to inactivate the SARS coronavirus (70). The PBMCs were then washed twice with fluorescence-activated cell sorting (FACS) buffer (PBS 2% FBS), spun down (400 g, 5 min, RT), and frozen in freezing media (90% FBS/10% DMSO) at −80° C. until FACs staining.

### Antibody Panels and Staining

On the day of FACs staining, PBMCs were thawed and washed in PBS 2% FBS (FACs buffer). PBMCs were stained with AF700-conjugated anti-CD3 (clone: UCHT1), APC-conjugated anti TCR alpha/beta (clone: IP26), FITC-conjugated anti TCR gamma/delta (clone: B1), BV785-conjugated anti-CD8 (clone: RPA-T8), BUV395-conjugated anti-CD4 (clone: RPA-T4), BV605-conjugated anti-PD-1 (clone: EH12.1), BV650-conjugated anti-Notch (clone: MHN1-519), and APC-Cy7-conjugated anti-CD69 (clone: FN50) in FACs buffer during 20 min at 4° C. in the dark. After that, cells were washed and fixed in Fixation/Permeabilization Buffer during 45 min at 4° C. in the dark and finally stained with PE-conjugated anti-TCF1 (clone: 7F11A10), PEcy7-conjugated anti-IFNg (clone: 4S.B3), PE-CF594-conjugated anti-Granzyme B (clone: GB11), FITC-conjugated anti-Ki67 (clone: 11F6) and BV421-conjugated anti-Bcl2 (clone: 100) during 30 min in Permeabilization Buffer. In caspase 3 staining cases, PBMCs were washed with FACS buffer and spun for 1700 rpm, 5 min before staining with FITC active Caspase-3 apoptosis kit then washed again (FACS buffer, 1700 rpm, 5 min, RT). UltraComp eBeads (ThermoFisher, catalog no. 01-2222-42) were used for compensation. The acquisition was performed by using BD LSRFortessa X-20 and DIVA software (Beckton Dickinson). FACS analyses were performed by using FlowJo software or Cytobank for viSNE analyses. The anti-TCR gamma/delta antibodies were a kind gift of Dr. Naglaa Shoukry (CR-CHUM, Montreal).

### Flow Cytometry and High-Dimensional Data Analysis

Samples were acquired on a five-laser BD FACS Symphony A5. Standardized SPHERO rainbow beads (Spherotech, catalog no. RFP-30-5A) were used to track and adjust photomultiplier tubes over time. UltraComp eBeads (ThermoFisher, catalog no. 01-2222-42) were used for compensation. Up to 2×10⁶ live PBMCs were acquired per sample. viSNE analyses were performed on Cytobank (cytobank.org). In this case, CD4 T cells, and CD8 T cells were analyzed separately. viSNE analysis was performed using equal sampling of 1000 cells from each FCS file, with 3000 iterations, a perplexity of 60, and a theta of 0.5. The following markers were used to generate the viSNE maps: CD8, CD4, CD44, TCF1, PD-1, CD69, CD45RA, CCR7, KLRG1, CD127, Ki67, IFNg, GzmB, Slamf6, Caspase3.

### Longitudinal Analysis and Correlation Plots

To assess samples from participants over time, Applicant sampled the same patients over a time after admission to the hospital and during the monitoring of the severity of disease. Pairwise correlations between variables were calculated and visualized as a correlogram using R function corrplot. Spearman's rank correlation coefficient (r) was indicated by square size and heat scale; significance was indicated by *P<0.05, **P<0.01, and ***P<0.001; and a black box indicates a false-discovery rate (FDR)<0.05.

### Quantitative Real-Time PCR

Total RNA was isolated from the PBMCs and reverse-transcribed to obtain cDNA using All-In-One 5×RT MasterMix (Applied Biological Materials Inc, BC, Canada). Real-time PCR amplification of the cDNA was analyzed using BrightGreen 2×qPCR MasterMix-Low ROX (Applied Biological Materials Inc, BC, Canada) and QS12K Flex (Bio-Rad Laboratories, Hercules, CA, USA) ABI QS12K Flex system (Thermo Fisher Scientific). The results were analyzed using the CFX Manager software and normalized to the levels of the housekeeping gene. The primers for qPCR analysis are shown, human-18S-F (GATTAAGTCCCTGCCCTTTGT) (SEQ ID NO: 1), human-18S-R (GTCAAGTTCGACCGTCTTCTC) (SEQ ID NO: 2), human-TCF7-F(CTGACCTCTCTGGCTTCTACTC) (SEQ ID NO: 3), human-TCF7-R(CAGAACCTAGCATCAAGGATGGG) (SEQ ID NO: 4), human-Bcl2-F(TGGATGACCGAGTACCTGAACCG) (SEQ ID NO: 5), and human-Bcl2-R(TGCCTTCAGAGACAGCCAGGAG) (SEQ ID NO: 6).

Isolation and Culture PBMCs with Human Sera and Anti-IL12

Fresh PBMCs were isolated from blood obtained from Hema-Quebec (Quebec) and rested overnight in RPMI 1640 medium (Corning, USA), 10% heat-inactivated fetal bovine serum (Gibco, USA), penicillin (100U/ml, Hyclone, USA), 2-Mercaptoethanol (50 μM, Sigma, USA), media at 37° C. On the second day, the PBMCs were stimulated with 10% healthy or severe sera, in the presence or absence of neutralization monoclonal antibody to IL12p40 (5 ug/ml, R&D) or Monoclonal Mouse IgG1 Clone #24901 isotype control antibody. After 6 days of culture, cells were resuspended in staining buffer (PBS with 2% FBS) and then stained with surface antibodies (anti-CD3, CD4, CD8) and intracellular antibody (anti-TCF1) for 30 min in the dark at 4° C. Cells were then washed twice with staining buffer and resuspended for analysis by a flow cytometer.

All data are expressed as mean±SEM. A t-test was used when only two groups were compared, and a one-way ANOVA was used when more than two groups were compared. A two-way ANOVA was used for multiple comparison procedures that involved two independent variables. A difference in mean values between groups was significant when p≤0.05*; p≤0.01** and p≤0.001***

### EQUIVALENTS

It is to be understood that while the disclosure has been described in conjunction with the above embodiments, that the foregoing description and examples are intended to illustrate and not limit the scope of the disclosure. Other aspects, advantages, and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scope of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. J. ter Meulen et al., Human monoclonal antibody combination against SARS coronavirus: synergy and coverage of escape mutants. PLOS Med 3, e237 (2006).
2. F. Amanat, F. Krammer, SARS-CoV-2 Vaccines: Status Report. Immunity 52, 583-589 (2020).
3. M. Z. Tay, C. M. Poh, L. Renia, P. A. MacAry, L. F. P. Ng, The trinity of COVID-19: immunity, inflammation and intervention. Nat Rev Immunol 20, 363-374 (2020).
4. F. Coperchini, L. Chiovato, L. Croce, F. Magri, M. Rotondi, The cytokine storm in COVID-19: An overview of the involvement of the chemokine/chemokine-receptor system. Cytokine Growth Factor Rev 53, 25-32 (2020).
5. C. Rydyznski Moderbacher et al., Antigen-Specific Adaptive Immunity to SARS-CoV-2 in Acute COVID-19 and Associations with Age and Disease Severity. Cell 183, 996-1012 e1019 (2020).
6. D. Weiskopf et al., Phenotype and kinetics of SARS-CoV-2-specific T cells in COVID-19 patients with acute respiratory distress syndrome. Sci Immunol 5 (2020).
7. A. Cizmecioglu et al., Apoptosis-Induced T Cell Lymphopenia Is Related to COVID-19 Severity. J Med Virol 10.1002/jmv.26742 (2020).
8. J. Braun et al., SARS-CoV-2-reactive T cells in healthy donors and patients with COVID-19. Nature 587, 270-274 (2020).
9. J. W. Song et al., Immunological and inflammatory profiles in mild and severe cases of COVID-19. Nat Commun 11, 3410 (2020).
10. S. Varchetta et al., Unique immunological profile in patients with COVID-19. Cell Mol Immunol 10.1038/s41423-020-00557-9 (2020).
11. B. Diao et al., Reduction and Functional Exhaustion of T Cells in Patients With Coronavirus Disease 2019 (COVID-19). Front Immunol 11, 827 (2020).
12. A. G. Laing et al., A dynamic COVID-19 immune signature includes associations with poor prognosis. Nat Med 26, 1623-1635 (2020).
13. D. Mathew et al., Deep immune profiling of COVID-19 patients reveals distinct immunotypes with therapeutic implications. Science 369 (2020).
14. N. Chen et al., Epidemiological and clinical characteristics of 99 cases of 2019 novel coronavirus pneumonia in Wuhan, China: a descriptive study. Lancet 395, 507-513 (2020).
15. G. Chen et al., Clinical and immunological features of severe and moderate coronavirus disease 2019. J Clin Invest 130, 2620-2629 (2020).
16. E. J. Giamarellos-Bourboulis et al., Complex Immune Dysregulation in COVID-19 Patients with Severe Respiratory Failure. Cell Host Microbe 27, 992-1000 e1003 (2020).
17. R. L. Chua et al., COVID-19 severity correlates with airway epithelium-immune cell interactions identified by single-cell analysis. Nat Biotechnol 38, 970-979 (2020).
18. A. Mazzoni et al., Impaired immune cell cytotoxicity in severe COVID-19 is IL-6 dependent. J Clin Invest 130, 4694-4703 (2020).
19. J. P. Bottcher et al., IL-6 trans-signaling-dependent rapid development of cytotoxic CD8+ T cell function. Cell Rep 8, 1318-1327 (2014).
20. R. de Waal Malefyt et al., Interleukin 10 (IL-10) and viral IL-10 strongly reduce antigen-specific human T cell proliferation by diminishing the antigen-presenting capacity of monocytes via downregulation of class II major histocompatibility complex expression. J Exp Med 174, 915-924 (1991).
21. Y. Liu et al., Neutrophil-to-lymphocyte ratio as an independent risk factor for mortality in hospitalized patients with COVID-19. J Infect 81, e6-e12 (2020).
22. C. Huang et al., Clinical features of patients infected with 2019 novel coronavirus in Wuhan, China. Lancet 395, 497-506 (2020).
23. L. Tan et al., Lymphopenia predicts disease severity of COVID-19: a descriptive and predictive study. Signal Transduct Target Ther 5, 33 (2020).
24. M. Liao et al., Single-cell landscape of bronchoalveolar immune cells in patients with COVID-19. Nat Med 26, 842-844 (2020).
25. D. Wichmann et al., Autopsy Findings and Venous Thromboembolism in Patients With COVID-19: A Prospective Cohort Study. Ann Intern Med 173, 268-277 (2020).
26 J. C. Melms et al., A molecular single-cell lung atlas of lethal COVID-19. Nature 595, 114-119 (2021).
27. T. M. Delorey et al., COVID-19 tissue atlases reveal SARS-CoV-2 pathology and cellular targets. Nature 595, 107-113 (2021).
28. X. Zhao, Q. Shan, H. H. Xue, TCF1 in T cell immunity: a broadened frontier. Nat Rev Immunol 10.1038/s41577-021-00563-6 (2021).
29. B. N. Weber et al., A critical role for TCF-1 in T-lineage specification and differentiation. Nature 476, 63-68 (2011).
30. C. Boudousquie et al., Differences in the transduction of canonical Wnt signals demarcate effector and memory CD8 T cells with distinct recall proliferation capacity. J Immunol 193, 2784-2791 (2014).
31. H. H. Chu et al., Continuous Effector CD8(+) T Cell Production in a Controlled Persistent Infection Is Sustained by a Proliferative Intermediate Population. Immunity 45, 159-171 (2016).
32. A. Delpoux et al., Continuous activity of Foxol is required to prevent anergy and maintain the memory state of CD8(+) T cells. J Exp Med 215, 575-594 (2018).
33. G. Jeannet et al., Essential role of the Wnt pathway effector Tcf-1 for the establishment of functional CD8 T cell memory. Proc Natl Acad Sci USA 107, 9777-9782 (2010).
34. D. T. Utzschneider et al., T Cell Factor 1-Expressing Memory-like CD8(+) T Cells Sustain the Immune Response to Chronic Viral Infections. Immunity 45, 415-427 (2016).
35. S. P. M. Welten et al., Tcf1(+) cells are required to maintain the inflationary T cell pool upon MCMV infection. Nat Commun 11, 2295 (2020).
36. S. J. Im et al., Defining CD8+ T cells that provide the proliferative burst after PD-1 therapy. Nature 537, 417-421 (2016).
37. Y. A. Leong et al., CXCR5(+) follicular cytotoxic T cells control viral infection in B cell follicles. Nat Immunol 17, 1187-1196 (2016).
38. T. Wu et al., The TCF1-Bcl6 axis counteracts type I interferon to repress exhaustion and maintain T cell stemness. Sci Immunol 1 (2016).
39. I. Siddiqui et al., Intratumoral Tcf1(+) PD-1(+) CD8(+) T cells with stem-like properties promote tumor control in response to vaccination and checkpoint blockade immunotherapy. Immunity 50, 195-211 e110 (2019).
40. S. Kurtulus et al., Checkpoint blockade immunotherapy induces dynamic changes in PD-1(−)CD8(+) tumor-infiltrating T cells. Immunity 50, 181-194 e186 (2019).
41. Z. Chen et al., TCF-1-Centered Transcriptional Network Drives an Effector versus Exhausted CD8 T Cell-Fate Decision. Immunity 51, 840-855 e845 (2019).
42. J. Gerdes et al., Cell cycle analysis of a cell proliferation-associated human nuclear antigen defined by the monoclonal antibody Ki-67. J Immunol 133, 1710-1715 (1984).
43. M. G. Davey, S. O. Hynes, M. J. Kerin, N. Miller, A. J. Lowery, Ki-67 as a Prognostic Biomarker in Invasive Breast Cancer. Cancers (Basel) 13 (2021).
44. R. J. Youle, A. Strasser, The BCL-2 protein family: opposing activities that mediate cell death. Nat Rev Mol Cell Biol 9, 47-59 (2008).
45. J. Behrens et al., Functional interaction of beta-catenin with the transcription factor LEF-1. Nature 382, 638-642 (1996).
46 Q. Shan et al., Tef1 and Lef1 provide constant supervision to mature CD8(+) T cell identity and function by organizing genomic architecture. Nat Commun 12, 5863 (2021).
47. A. Sambandam et al., Notch signaling controls the generation and differentiation of early T lineage progenitors. Nat Immunol 6, 663-670 (2005).
48. T. R. Burglin, M. Affolter, Homeodomain proteins: an update. Chromosoma 125, 497-521 (2016).
49 U. Wong, R. K. Cross, Expert opinion on interleukin-12/23 and interleukin-23 antagonists as potential therapeutic options for the treatment of inflammatory bowel disease. Expert Opin Investig Drugs 28, 473-479 (2019).
50. R. C. Group et al., Dexamethasone in Hospitalized Patients with Covid-19. N Engl J Med 384, 693-704 (2021).
51. W. Ma et al., Dexamethasone inhibits IL-12p40 production in lipopolysaccharide-stimulated human monocytic cells by down-regulating the activity of c-Jun N-terminal kinase, the activation protein-1, and NF-kappa B transcription factors. J Immunol 172, 318-330 (2004).
52. A. D. Amir el et al., viSNE enables visualization of high dimensional single-cell data and reveals phenotypic heterogeneity of leukemia. Nat Biotechnol 31, 545-552 (2013).
53. WHO, COVID-19 Therapeutic Trial Synopsis. World Health Organization (2020).
54. K. Bugin, J. Woodcock, Trends in COVID-19 therapeutic clinical trials. Nat Rev Drug Discov 20, 254-255 (2021).
55. C. Guo et al., Single-cell analysis of two severe COVID-19 patients reveals a monocyte-associated and tocilizumab-responding cytokine storm. Nat Commun 11, 3924 (2020).
56. A. Tarke et al., Comprehensive analysis of T cell immunodominance and immunoprevalence of SARS-CoV-2 epitopes in COVID-19 cases. bioRxiv 10.1101/2020.12.08.416750 (2020).
57. A. Grifoni et al., Targets of T Cell Responses to SARS-CoV-2 Coronavirus in Humans with COVID-19 Disease and Unexposed Individuals. Cell 181, 1489-1501 e1415 (2020).
58. A. A. Seyhan et al., Novel biomarkers of a peripheral blood interferon signature associated with drug-naive early arthritis patients distinguish persistent from self-limiting disease course. Sci Rep 10, 8830 (2020).
59. S. M. Kaech, W. Cui, Transcriptional control of effector and memory CD8+ T cell differentiation. Nat Rev Immunol 12, 749-761 (2012).
60. J. Krueger et al., Hydroxychloroquine (HCQ) decreases the benefit of anti-PD-1 immune checkpoint blockade in tumor immunotherapy. PLOS One 16, e0251731 (2021).
61. N. S. Joshi et al., Inflammation directs memory precursor and short-lived effector CD8(+) T cell fates via the graded expression of T-bet transcription factor. Immunity 27, 281-295 (2007).
62. S. Sarkar et al., Functional and genomic profiling of effector CD8 T cell subsets with distinct memory fates. J. Exp. Med. 205, 625-640 (2008).
63. D. Herndler-Brandstetter et al., KLRG1(+) effector CD8(+) T cells lose KLRG1, differentiate into all memory T cell lineages, and convey enhanced protective immunity. Immunity 48, 716-729 e718 (2018).
64. P. O. Barros, T. K. Berthoud, N. Aloufi, J. B. Angel, Soluble IL-7Ralpha/sCD127 in Health, Disease, and Its Potential Role as a Therapeutic Agent. Immunotargets Ther 10, 47-62 (2021).
65. P. L. Schwartzberg, K. L. Mueller, H. Qi, J. L. Cannons, SLAM receptors and SAP influence lymphocyte interactions, development and function. Nat Rev Immunol 9, 39-46 (2009).
66. D. M. Del Valle et al., An inflammatory cytokine signature predicts COVID-19 severity and survival. Nat Med 26, 1636-1643 (2020).
67. M. Danilo, V. Chennupati, J. G. Silva, S. Siegert, W. Held, Suppression of Tcf1 by Inflammatory Cytokines Facilitates Effector CD8 T Cell Differentiation. Cell Rep 22, 2107-2117 (2018).
68. Y. Peng et al., Broad and strong memory CD4(+) and CD8(+) T cells induced by SARS-CoV-2 in UK convalescent individuals following COVID-19. Nat Immunol 21, 1336-1345 (2020).
69. B. J. Meckiff et al., Imbalance of Regulatory and Cytotoxic SARS-CoV-2-Reactive CD4(+) T Cells in COVID-19. Cell 183, 1340-1353 e1316 (2020).
70. R. Kratchmarov, A. M. Magun, S. L. Reiner, TCF1 expression marks self-renewing human CD8(+) T cells. Blood Adv 2, 1685-1690 (2018).
71. M. Eltobgy, A. Zani, A. D. Kenney, et al., Caspase-4/11 exacerbates disease severity in SARS-CoV-2 infection by promoting inflammation and thrombosis. bioRxiv doi.org/10.1101/2021.09.24.461743 (2021).
72. Q. Yu et al., T cell factor 1 initiates the T helper type 2 fate by inducing the transcription factor GATA-3 and repressing interferon-gamma. Nat Immunol 10, 992-999 (2009).
73. J. A. Walker, A. N. J. Mckenzie, TH2 cell development and function. Nat Rev Immunol 18, 121-133 (2018).

1. A method of determining whether a subject suffering from an acute respiratory syndrome coronavirus infection is more likely to suffer from severe or mild disease comprising detecting the amount of transcription factor TCF1+ (T-cell factor-1) and in addition, any one or more chosen from: Ki67, LEF1, Notch1, Eomes, Bcl2 or Caspase 3 in T cells in a biological sample comprising T cells that were isolated from the subject and comparing a measured amount to a reference amount, wherein a modified measured amount compared to the reference amount is indicative that the subject will suffer from severe or mild SARS, and optionally wherein the coronavirus is a SARS-Cov-2 coronavirus infection.

2. The method of claim 1, wherein the amount of SLAMF6 is detected instead of TCF1+ and further wherein the detected amount of SLAMF6 is a surrogate for the detected amount of TCF1+.

3. The method of claim 1, wherein the modified measured amount is an increase in the measured amount.

4. The method of claim 1, wherein the modified measured amount is a decrease in the measured amount.

5. The method of claim 3, wherein the increase in the measured amount is indicative that the subject is more likely to suffer from mild disease.

6. The method of claim 4, wherein the decrease in the measured amount compared to the reference amount is indicative that the subject is more likely to suffer from severe disease.

7. The method of claim 1, wherein the T-cell is selected from a CD4+ or a CD8+ T cell.

8. The method of claim 1 wherein severe SARS disease has a World Health Organization (WHO) severity score of 5 or higher.

9. The method of claim 1, wherein mild SARS disease has a World Health Organization (WHO) severity score of 4 or less.

10. The method of claim 1, wherein the patient is clinically lymphopenic.

11. The method of claim 1, wherein the biological sample comprises peripheral blood lymphocytes (PBMCs).

12. The method of claim 1, further comprising isolating the biological sample is taken from the subject prior to the comparison.

13. The method of claim 1, further comprising taking a further biological sample from the subject after treatment to the detecting step.

14. The method of claim 1, further comprising administering a therapy to treat severe SARS.

15. The method of claim 14, wherein the therapy is selected from an anti-IL 12 therapy or an antibody cocktail therapy, wherein the anti-IL-12 therapy comprises an anti-IL-12 antibody therapy.

16. (canceled)

17. The method of claim 15, wherein the anti-IL-12 antibody therapy comprises ustekinumab (Stelara).

18. The method of claim 14, wherein the therapy comprises an anti-inflammatory glucocorticoid, optionally dexamethasone.

19. (canceled)

20. A method for treating acute respiratory syndrome coronavirus 2 (SARS-Cov-2 or SARS) infection and having a reduced amount of transcription factor TCF1+ (T-cell factor-1) expressing T cells comprising administering an effective amount of a therapy to treat severe SARS disease, and optionally wherein the coronavirus 2 (SARS-Cov-2 or SARS) infection.

21.-25. (canceled)

26. A method of predicting whether a subject suffering from an acute respiratory syndrome coronavirus infection with mild disease will progress to severe disease and possible death by detecting the expression of one or a combination of markers selected from: transcription factor TCF1, transcription factor EOMES, the surrogate cell surface marker for TCF1 termed SLAMF6, the proliferation marker Ki67, the pro-survival marker BcL-2 and the marker for cell death Caspase 3 in T cells in a biological sample comprising T cells that were isolated from the subject and comparing a measured amount to a reference amount, wherein a modified measured amount compared to the reference amount is indicative that the subject will suffer from severe or mild SARS disease, and optionally wherein the coronavirus 2 (SARS-Cov-2 or SARS) infection.

27.-42. (canceled)

43. A method for monitoring disease state in a subject suffering from an acute respiratory syndrome coronavirus infection comprising detecting the amount of transcription factor TCF1+ (T-cell factor-1) and in addition, any one or more chosen from: Ki67, LEF1, Notch1, Eomes, Bcl2 or Caspase 3 in T cells in a biological sample comprising T cells that were isolated from the subject and comparing a measured amount to a reference amount, wherein a modified measured amount compared to the reference amount is indicative of the status of the coronavirus infection.

44.-54. (canceled)
